(19) United States
(12) Patent Application Publication
(10) Pub. No.: US 2004/0186083 A1
(43) Pub. Date: Sep. 23, 2004

(54) Combination of an aldosterone receptor antagonist and an endothelin receptor antagonist and/or endothelin converting enzyme inhibitor

(75) Inventors: Ellen G. McMahon, St. Louis, MO (US); Amy E. Rudolph, Fairfield, CT (US)

Correspondence Address: SENNIGER POWERS LEAVITT AND ROEDEL, ONE METROPOLITAN SQUARE, 16TH FLOOR, ST LOUIS, MO, 63102, US

(73) Assignee: Pharmacia Corporation

(21) Appl. No.: 10/803,818

(22) Filed: Mar. 18, 2004

Related U.S. Application Data
(60) Provisional application No. 60/455,580, filed on Mar. 18, 2003.

(51) Int. Cl. A61K 31/56

(52) U.S. Cl. 514/171

(57) ABSTRACT

Novel combinations, compositions, and therapeutic methods of treatment and/or prophylaxis of a hypertension, cardiovascular disease, renal dysfunction, edema, cerebrovascular disease, or insulinopathy pathological conditions in a subject, wherein the methods comprise the administration of a combination of one or more aldosterone receptor antagonists and one or more endothelin receptor antagonist and/or ECE inhibitors selected from a specific group of compounds described herein.

### FIELD OF THE INVENTION

Combinations of an aldosterone receptor antagonist and an endothelin receptor antagonist and/or an endothelin converting enzyme inhibitor, compositions thereof, and therapeutic methods are described for use in the treatment of pathological conditions.

### BACKGROUND OF THE INVENTION

Aldosterone is the body's most potent known mineralocorticoid hormone. As connoted by the term mineralocorticoid, this steroid hormone has mineral-regulating activity. It promotes sodium (Na⁺) reabsorption in epithelial cells through binding and activating the mineralocorticoid receptor (MR). Aldosterone increases sodium and water reabsorption in the distal nephron and promotes potassium (K⁺) and magnesium (Mg²⁺) excretion.

Aldosterone also can produce responses in nonepithelial cells. In fact, aldosterone receptors have been recently identified in brain tissue, heart tissue and blood vessels. These aldosterone-mediated responses can have adverse consequences on the structure and function of the cardiovascular system. Hence, inappropriate aldosterone exposure can contribute to organ damage in disease settings.

The effect of aldosterone can be reduced through the use of an aldosterone receptor antagonist. Spironolactone, also known as ALDACTONE® (Pharmacia, Chicago, Ill.), is an example of an aldosterone receptor antagonist. According to United States Pharmacopeia, Rockville, Md., spironolactone is indicated for the management of essential hypertension, primary aldosteronism, hypokalemia, and edematous conditions such as congestive heart failure, cirrhosis of the liver, and nephrotic syndrome. The administration of spironolactone to severe heart failure patients was evaluated in the Randomized Aldactone Evaluation Study (RALES). RALES was a randomized, double-blinded, placebo-controlled trial that enrolled participants who had severe heart failure and a left ventricular ejection fraction of no more than 35% and who were receiving standard therapy, which typically included an angiotensin-converting enzyme inhibitor, a loop diuretic, and, in some cases, digoxin. The RALES subjects treated with spironolactone had a statistically significant reduction in mortality and incidence of hospitalization relative to placebo-treated subjects. New England Journal of Medicine 341, 709-717 (1999).

A class of steroidal-type aldosterone receptor antagonists exemplified by epoxy-containing spirolactone derivatives is described in U.S. Pat. No. 4,559,332 issued to Grob et al. This patent describes 9α,11α-epoxy-containing spirolactone derivatives as aldosterone receptor antagonists that are useful for the treatment of hypertension, cardiac insufficiency and cirrhosis of the liver. One of the epoxy-steroidal aldosterone receptor antagonist compounds described in U.S. Pat. No. 4,559,332 is eplerenone (also known as epoxymexrenone). Eplerenone is an aldosterone receptor antagonist that has a higher specificity for the MR compared to spironolactone.

Endothelin (ET) is a peptide which is composed of 21 amino acids that is synthesized and released by the vascular endothelium. Endothelin is produced by enzymatic cleavage of a Trp-Val bond in the precursor peptide big endothelin (Big ET). This cleavage is caused by an endothelin converting enzyme (ECE). Endothelin exists as three isoforms, ET-1, ET-2 and ET-3 (hereinafter, unless otherwise stated, “endothelin” shall mean any or all of the isoforms of endothelin). Endothelin acts on two pharmacologically distinct subtypes of receptors, termed ET_{A} and ET_{B}, that are expressed on a wide variety of vascular and non-vascular target cells, eliciting, for example, contraction and proliferation of vascular smooth muscle cells and release of nitric oxide from endothelial cells.

Endothelin is associated with smooth muscle contraction which is involved in the pathogenesis of, inter alia, cardiovascular, cerebrovascular, respiratory and renal pathophysiology. It has been shown, among other things, to constrict arteries and veins, increase mean arterial blood pressure, decrease cardiac output, increase cardiac contractility in vitro, stimulate mitogenesis in vascular smooth muscle cells in vitro, stimulate release of atrial natriuretic factor in vitro and in vivo, increase plasma levels of vasopressin, aldosterone and catecholamines, inhibit release of renin in vitro and stimulate release of gonadotropins in vitro.

An agent which suppresses endothelin production, such as an ECE inhibitor, or which inhibits the binding of endothelin to an endothelin receptor, such as an endothelin receptor antagonist, antagonizes various physiological effects of endothelin and produces beneficial effects in a variety of therapeutic areas. Endothelin receptor antagonists and ECE inhibitors are therefore useful in treating a variety of diseases affected by endothelin. A non-exhaustive list of such diseases includes chronic heart failure, myocardial infarction, cardiogenic shock, systemic and pulmonary hypertension, ischemia-repurfusion injury, atherosclerosis, coronary and systemic vasospastic disorders, cerebral vasospasm, and subarachnoid hemorrhage and the like.

Therapies comprising the administration of an aldosterone receptor antagonist in combination with several other pharmacologically active compounds have been reported in the literature.

Egan et al., WO 96/40255, disclose a combination treatment therapy utilizing a an epoxy-steroidal aldosterone receptor antagonist and an angiotensin II antagonist for treating cardiofibrosis.

Alexander et al., WO 96/40257, disclose a combination treatment therapy utilizing an epoxy-steroidal aldosterone receptor antagonist and an angiotensin II antagonist for treating congestive heart failure.

Williams et al., WO 01/95892 and WO 01/95893, describe methods for the treatment of aldosterone-mediated pathogenic effects in a subject using an aldosterone receptor antagonist (including spironolactone and/or eplerenone).

Rocha et al., WO 02/09683, describe methods of using an aldosterone receptor antagonist (including eplerenone and/or spironolactone) for the treatment of inflammation in a subject.

Perez et al., WO 00/27380, disclose a combination treatment therapy utilizing an angiotensin converting enzyme inhibitor and an aldosterone receptor antagonist for reducing morbidity and mortality resulting from cardiovascular disease.

Alexander et al., WO 00/51642, disclose a combination treatment therapy utilizing an angiotensin converting enzyme inhibitor and an epoxy-steroidal aldosterone receptor antagonist for treating cardiovascular disease.

Alexander et al., WO 02/09760, disclose a combination therapy utilizing an epoxy-steroidal aldosterone receptor antagonist and a beta-adrenergic antagonist for treating circulatory disorders, including cardiovascular disorders such as hypertension, congestive heart failure, cirrhosis and ascites.

Schuh, WO 02/09761, discloses a combination treatment therapy utilizing an epoxy-steroidal aldosterone receptor antagonist and a calcium channel blocker for treating hypertension, congestive heart failure, cirrhosis and ascites.

Rocha et al., WO 02/09759, disclose a combination treatment therapy utilizing an epoxy-steroidal aldosterone receptor antagonist and a cyclooxygenase-2 inhibitor for treating inflammation related cardiovascular disorders.

Improved drug therapies for the treatment of subjects suffering from or susceptible to a pathological condition are highly desirable. In particular, there still is a need for drug therapies that (1) provide better control over pathological conditions, (2) further reduce pathological risk factors, (3) provide improved treatment and/or prevention of pathological conditions, (4) are effective in a greater proportion of subjects suffering from or susceptible to a pathological condition, particularly in those subjects who do not satisfactorily respond to conventional drug therapies, and/or (5) provide an improved side-effect profile relative to conventional drug therapies.

### SUMMARY OF THE INVENTION

The present invention is directed to a method for the prophylaxis or treatment of a pathological condition in a subject, which comprises administering an aldosterone receptor antagonist and an endothelin receptor antagonist for the prophylaxis or treatment of a pathological condition.

The invention is further directed to a combination comprising an aldosterone receptor antagonist and an endothelin receptor antagonist.

The invention is further directed to a method for the prophylaxis or treatment of a pathological condition in a subject, which comprises administering an aldosterone receptor antagonist and an ECE inhibitor for the prophylaxis or treatment of a pathological condition.

The invention is further directed to a combination comprising an aldosterone receptor antagonist and an ECE inhibitor.

The invention is further directed to a pharmaceutical composition comprising a first amount of an aldosterone receptor antagonist, a second amount of an endothelin receptor antagonist, and a pharmaceutically acceptable carrier.

The invention is further directed to a pharmaceutical composition comprising a first amount of an aldosterone receptor antagonist, a second amount of an ECE inhibitor, and a pharmaceutically acceptable carrier.

The invention is further directed to a kit containing a first amount of an aldosterone receptor antagonist and a second amount of an endothelin receptor antagonist.

The invention is further directed to a kit containing a first amount of an aldosterone receptor antagonist and a second amount of an ECE inhibitor.

Other aspects of the invention will be in part apparent and in part pointed out hereinafter.

### DETAILED DESCRIPTION OF THE PREFERRED EMBODIMENTS

The present invention relates to combinations, compositions, and methods to treat or prevent one or more pathological conditions in a subject through the therapeutical administration of an aldosterone receptor antagonist in combination with an ECE inhibitor and/or an endothelin receptor antagonist.

In one embodiment, the aldosterone receptor antagonist is an epoxy-steroidal aldosterone receptor antagonist. In another embodiment, the aldosterone receptor antagonist is an epoxy-steroidal aldosterone receptor antagonist containing a 9,11-epoxy moiety. In still another embodiment, the aldosterone receptor antagonist is Pregn-4-ene-7,21-dicarboxylic acid, 9,11-epoxy-17-hydroxy-3-oxo-,γ-lactone, methyl ester, (7α,11α,17α)—(also known as eplerenone or epoxymexrenone).

In another embodiment, the aldosterone receptor antagonist is a spirolactone-type aldosterone receptor antagonist, such as spironolactone.

In another embodiment, the aldosterone receptor antagonist is selected from the group consisting of eplerenone and spironolactone. In another embodiment, the aldosterone receptor antagonist is eplerenone.

In another embodiment, the method comprises the therapeutical administration of an aldosterone receptor antagonist in combination with an ECE inhibitor and an endothelin receptor antagonist. In another embodiment, the aldosterone receptor antagonist is selected from the group consisting of eplerenone and spironolactone. In still another embodiment, the aldosterone receptor antagonist is eplerenone.

Indications

The pathological conditions that can be treated or prevented in accordance with the present invention include, but are not limited to, hypertension, cardiovascular disease, renal dysfunction, liver disease, cerebrovascular disease, vascular disease, pulmonary dysfunction, retinopathy, neuropathy (such as peripheral neuropathy), organ damage, insulinopathy, edema, endothelial dysfunction, baroreceptor dysfunction, migraine headaches, hot flashes, premenstrual tension, glaucoma, diabetes, Buerger's Disease, Crohn's Disease, plasma levels of vasopressin, aldosterone and catecholamines, the release of renin, and the like. Cardiovascular disease includes, but is not limited to, heart failure, congestive heart failure, cardiac hypertrophy, arrhythmia, diastolic dysfunction (such as left ventricular diastolic dysfunction, diastolic heart failure, and impaired diastolic filling), systolic dysfunction, ischemia, ischemia-repurfusion injury, hypertrophic cardiomyopathy, sudden cardiac death, myocardial and vascular fibrosis, restinosis after angioplasty, myocardial dysfunction during or following a myocardial infarction, impaired arterial compliance, myocardial necrotic lesions, vascular damage, myocardial infarction, left ventricular hypertrophy, decreased ejection fraction, cardiac lesions, vascular wall hypertrophy, endothelial thickening, fibrinoid necrosis of coronary arteries, and the like. Renal dysfunction includes, but is not limited to, renal failure, glomerulosclerosis, end-stage renal disease, renal impairment following treatment with cyclosporine or other immunosuppressants diabetic nephropathy, reduced renal blood flow, increased glomerular filtration fraction, proteinuria, decreased glomerular filtration rate, decreased creatinine clearance, microalbuminuria, renal arteriopathy, ischemic lesions, thrombotic lesions, global fibrinoid necrosis, focal thrombosis of glomerular capillaries, swelling and proliferation of intracapillary (endothelial and mesangial) and/or extracapillary cells (crescents), expansion of reticulated mesangial matrix with or without significant hypercellularity, malignant nephrosclerosis (such as ischemic retraction, thrombonecrosis of capillary tufts, arteriolar fibrinoid necrosis, and thrombotic microangiopathic lesions of affecting glomeruli and microvessels), and the like. Liver disease includes, but is not limited to, liver cirrhosis, liver ascites, hepatic congestion, and the like. Cerebrovascular disease includes stroke, cerebral vasospasm, cerebral infarction and neuronal death, cerebral ischemia, stroke, and cerebral ischemia, subarachnoid hemorrhage, and the like. Vascular disease includes, but is not limited to, thrombotic vascular disease (such as mural fibrinoid necrosis, extravasation and fragmentation of red blood cells, and luminal and/or mural thrombosis), proliferative arteriopathy (such as swollen myointimal cells surrounded by mucinous extracellular matrix and nodular thickening), atherosclerosis, decreased vascular compliance (such as stiffness, reduced ventricular compliance and reduced vascular compliance), endothelial dysfunction, proliferation of vascular smooth muscle cells, systemic vasospastic disorders, vascular wall hypertrophy, Raynaud's syndrome, mitogenesis, Takayasu's arteritis, atherosclerosis, and the like. Pulmonary dysfunction includes, but is not limited to, pulmonary hypertension, increased airway resistance, asthma, hyperplasia and intimal fibrosis of cryptogenic fibrosing alveolitis, acute respiratory distress syndrome (ARDS), severe apnea, ischemic lesions, and chronic obstructive pulmonary diseases, and the like. Edema includes, but is not limited to, peripheral tissue edema, hepatic congestion, splenic congestion, liver ascites, respiratory or lung congestion, and the like. Insulinopathies include, but are not limited to, insulin resistance, Type I diabetes mellitus, Type II diabetes mellitus, glucose sensitivity, pre-diabetic state, syndrome X, and the like. Gastroenteric disorders such as diarrhea and hyperchlorhydria, irritable bowel syndrome. Endocrine and metabolic disease such as obesity hyperaldosteronemia, glaucoma, hypertensive or diabetic retinopathy, elevated intraocular pressure, and the like. Autoimmune disease such as rheumatism.

The pathological conditions described above are hereinafter individually and collectively described as “pathological conditions.”

In one embodiment the pathological condition is selected from the group consisting of hypertension, cardiovascular disease, renal dysfunction, edema, cerebrovascular disease, insulinopathies, and combinations thereof.

In another embodiment the pathological condition is selected from the group consisting of hypertension, cardiovascular disease, stroke, Type II diabetes mellitus, and combinations thereof.

In another embodiment the pathological condition is selected from the group consisting of hypertension, heart failure (particularly heart failure post myocardial infarction), left ventricular hypertrophy, stroke, and combinations thereof.

In another embodiment the pathological condition is hypertension.

In another embodiment the pathological condition is heart failure.

In another embodiment the pathological condition is myocardial infarction.

In another embodiment the pathological condition is stroke.

In another embodiment the pathological condition is atherosclerosis.

In another embodiment the pathological condition is renal dysfunction.

In another embodiment the pathological condition is organ damage.

In another embodiment the pathological condition is diabetes.

Subjects in Need of Treatment or Prevention

In addition to being suitable for human use, the present combination therapy is also suitable for treatment of animals, including mammals such as horses, dogs, cats, rats, mice, sheep, pigs, and the like.

The pathogenicity of endogenous aldosterone at a sub-normal level in human subjects was not previously appreciated. Similarly, the increased development, rapidity of onset and development, and/or severity of pathological conditions meditated by endogenous aldosterone in a human caused by the presence of elevated sodium levels previously was not appreciated. It is conventionally believed that sodium loading typically results in a decrease of endogenous aldosterone levels to non-pathogenic levels. Accordingly, subjects who can benefit from treatment or prophylaxis in accordance with the present invention are generally human subjects who have (i) a sub-normal endogenous aldosterone level, (ii) salt sensitivity regardless of the endogenous aldosterone level, and/or (iii) elevated dietary sodium intake regardless of the endogenous aldosterone level. Within each of these groups of subjects, it can be beneficial to carry out further profiling and/or phenotyping to identify sub-groups of subjects who will benefit from the therapy of the present invention.

Accordingly, in one embodiment, the subject benefitting from the treatment or prophylaxis in accordance with the method of the present invention are human subjects generally exhibiting one or more of the following characteristics:

(a) The average daily intake of sodium chloride by the subject is at least about 4 grams, particularly where this condition is satisfied over any one month interval for at least one or more monthly intervals over a given annual period. The average daily intake of sodium by the subject is at least about 6 grams. In another embodiment, the average daily intake of sodium by the subject is at least about 8 grams. In still another embodiment, the average daily intake of sodium by the subject is at least about 12 grams.

(b) In one embodiment, the subject exhibits an increase in systolic blood pressure and/or diastolic blood pressure of at least about 5%, when daily sodium chloride intake by the subject is increased from less than about 3 g/day to at least about 10 g/day. In another embodiment, the subject exhibits an increase in systolic blood pressure and/or diastolic blood pressure of at least about 7%. In still another embodiment, the subject exhibits an increase in systolic blood pressure and/or diastolic blood pressure of at least about 10%.

(c) In another embodiment, the activities ratio of plasma aldosterone (ng/dL) to plasma renin (ng/ml/hr) in the subject is greater than about 30. In another embodiment, the activities ratio is greater than about 40. In another embodiment, the activities ratio is greater than about 50. In still another embodiment, the activities ratio is greater than about 60.

(d) The subject has low plasma renin levels; for example, the morning plasma renin activity in the subject is less than about 1.0 ng/dL/hr, and/or the active renin value in the subject is less than about 15 pg/mL.

(e) The subject suffers from or is susceptible to elevated systolic and/or diastolic blood pressure. In one embodiment, the systolic blood pressure (measured, for example, by seated cuff mercury sphygmomanometer) of the subject is at least about 130 mm Hg. In another embodiment, the systolic blood pressure is at least about 140 mm Hg. In still another embodiment, the systolic blood pressure is at least about 150 mm Hg. Examples of elevated diastolic blood pressure (measured, for example, by seated cuff mercury sphygmomanometer) include at least about 85 mm Hg, at least about 90 mm Hg, and at least about 100 mm Hg.

(f) The urinary sodium to potassium ratio (mmol/mmol) of the subject is less than about 6; less than about 5.5; less than about 5; or less than about 4.5.

(g) The urinary sodium level of the subject is at least 60 mmol per day, particularly where this condition is satisfied over any one month interval for at least one or more monthly intervals over a given annual period. In another embodiment, the urinary sodium level of the subject is at least about 100 mmol per day. In another embodiment, at least about 150 mmol per day. In still another embodiment, at least about 200 mmol per day.

(h) The plasma concentration of one or more endothelins, particularly plasma immunoreactive ET-1, in the subject is elevated. Examples include plasma concentrations of ET-1 greater than about 2.0 pmol/L, greater than about 4.0 pmol/L, and greater than about 8.0 pmol/L.

(i) The subject has blood pressure that is substantially refractory to treatment with an ACE inhibitor; examples include a subject whose blood pressure is lowered less than about 8 mm Hg, less than 5 mm Hg, and less than 3 mm Hg, in response to 10 mg/day enalapril compared to the blood pressure of the subject on no antihypertensive therapy.

(j) The subject has blood volume-expanded hypertension or blood volume-expanded borderline hypertension, that is, hypertension wherein increased blood volume as a result of increased sodium retention contributes to blood pressure.

(k) The subject is a non-modulating individual, that is, the individual demonstrates a blunted positive response in renal blood flow rate and/or in adrenal production of aldosterone to an elevation in sodium intake or to angiotensin II administration, particularly when the response is less than the response of individuals sampled from the general geographical population (for example, individuals sampled from the subject's country of origin or from a country of which the subject is a resident). Examples include when the response is less than 40% of the mean of the population; when the response is less than 30%; and when the response is still less than 20%.

(l) The subject has or is susceptible to renal dysfunction, particularly renal dysfunction selected from one or more members of the group consisting of reduced glomerular filtration rate, microalbuminuria, and proteinuria.

(m) The subject has or is susceptible to cardiovascular disease, particularly cardiovascular disease selected from one or more members of the group consisting of heart failure, left ventricular diastolic dysfunction, hypertrophic cardiomyopathy, and diastolic heart failure.

(n) The subject has or is susceptible to liver disease, particularly liver cirrhosis. (o) The subject has or is susceptible to edema, particularly edema selected from one or more members of the group consisting of peripheral tissue edema, hepatic or splenic congestion, liver ascites, and respiratory or lung congestion.

(p) The subject has or is susceptible to insulin resistance, particularly Type I or Type II diabetes mellitus, and/or glucose sensitivity.

(q) In one embodiment, the subject is at least 55 years of age. In another embodiment, at least about 60 years of age. In still another embodiment, at least about 65 years of age. The subject is, in whole or in part, a member of at least one ethnic group selected from the Asian (particularly from the Japanese) ethnic group, the American Indian ethnic group, and the African ethnic group.

(r) The subject has one or more genetic markers associated with salt sensitivity.

(s) The subject is obese. Examples include subjects having greater than 25% body fat; greater than 30% body fat; and greater than 35% body fat.

(t) The subject has one or more 1^{st}, 2^{nd}, or 3^{rd} degree relatives who are or were salt sensitive, wherein 1^{st} degree relatives means parents or relatives sharing one or more of the same parents, 2^{nd} degree relatives means grandparents and relatives sharing one or more of the same grandparents, and 3^{rd} degree relatives means great-grandparents and relatives sharing one or more of the same great-grandparents. In one embodiment, individuals who have four or more salt sensitive 1^{st}, 2^{nd}, or 3^{rd} degree relatives. In another embodiment, eight or more such relatives. In another embodiment, 16 or more such relatives. In still another embodiment, individuals who have 32 or more such relatives.

Unless otherwise indicated to the contrary, the values listed above represent an average value. In another embodiment, the values listed above represent a daily average value based on at least two measurements.

In one embodiment, the subject in need of treatment satisfies at least two or more of the above-characteristics. In another embodiment, the subject in need of treatment satisfies at least three or more of the above-characteristics. In still another embodiment, the subject in need of treatment satisfies at least four or more of the above-characteristics.

In another embodiment of the present invention, the method comprises administering a therapeutically-effective amount of one or more aldosterone receptor antagonist compounds to treat or prevent one or more aldosterone-mediated pathological conditions in a human subject suffering from or susceptible to pathological conditions, wherein the subject has a sub-normal endogenous aldosterone level. In one embodiment, the epoxy-steroidal compound is selected from the group consisting of spironolactone and eplerenone. In another embodiment, the epoxy-steroidal compound is eplerenone. In another embodiment, the subject of the treatment or prophylaxis preferably is an individual having salt sensitivity and/or an elevated dietary sodium intake.

Accordingly, in one embodiment of the present invention the subject in need of treatment is salt sensitive and satisfies two or more of the following conditions: (i) the average daily intake of sodium chloride by the subject is at least about 4 grams, particularly where this condition is satisfied over any one month interval for at least one or more monthly intervals over a given annual period; and/or (ii) the activities ratio of plasma aldosterone (ng/dL) to plasma renin (ng/ml/hr) in the subject is greater than about 30; (iii) the morning plasma renin activity in the subject is less than about 1.0 ng/dL/hr, and/or the active renin value in the subject is less than about 15 pg/mL; and/or (iv) the systolic blood pressure of the subject is at least about 130 mm Hg and the diastolic blood pressure of the subject is at least about 85 mm Hg; and/or (v) the subject has or is susceptible to cardiovascular disease, particularly cardiovascular disease selected from one or more members of the group consisting of heart failure, left ventricular diastolic dysfunction, hypertrophic cardiomyopathy, and diastolic heart failure. In another embodiment of the present invention, the subject in need of treatment is salt sensitive and satisfies the following conditions: (i) the activities ratio of plasma aldosterone (ng/dL) to plasma renin (ng/ml/hr) in the subject is greater than about 30; and (ii) the morning plasma renin activity in the subject is less than about 1.0 ng/dL/hr, and/or the active renin value in the subject is less than about 15 pg/mL.

In another embodiment of the present invention, the subject in need of treatment is salt sensitive and satisfies at least two of the following conditions: (i) the average daily intake of sodium chloride by the subject is at least about 4 grams, particularly where this condition is satisfied over any one month interval for at least one or more monthly intervals over a given annual period; and/or (ii) the systolic blood pressure of the subject is at least about 130 mm Hg and the diastolic blood pressure of the subject is at least about 85 mm Hg; and/or (iii) the subject has or is susceptible to cardiovascular disease, particularly cardiovascular disease selected from one or more members of the group consisting of heart failure, left ventricular diastolic dysfunction, hypertrophic cardiomyopathy, ischemic heart disease, and diastolic heart failure.

Mechanism of Action

Without being held to a specific mechanism of action for the present combination therapy, it is hypothesized that the administration of these selected aldosterone receptor antagonists in combination with endothelin receptor antagonists and/or ECE inhibitors is effective because of the distinct physiological effects and pathways of the drugs as well as the simultaneous and interrelated responses of these two distinct classes of drugs on one or more target disorders. The combination is further hypothesized to be effective because of the effect of aldosterone receptor antagonists, endothelin receptor antagonists, and ECE inhibitors have on the biochemical feedback pathways that affect the regulation and release of aldosterone and other compounds in the body.

For purposes of illustration, in treating hypertension, aldosterone receptor antagonists block aldosterone from promoting the retention of sodium in the body. By blocking aldosterone, fluid retention is reduced and blood pressure levels are lowered. Endothelin receptor antagonists, utilizing a different pathway, inhibit endothelin from eliciting vasoconstriction. This enhances vasodialation which further reduces blood pressure. ECE inhibitors, by inhibiting ECE from catalyzing the formation of endothelin from Big-ET, also reduce the vasoconstriction resulting from circulating levels of endothelin. In addition to its vasoconstrictive properties, endothelin also enhances the release of aldosterone.

By administering an endothelin receptor antagonist, the further release of aldosterone is reduced inhibiting subsequent retention of fluids. As a result of the different pathways and the interrelationships of regulating aldosterone and other compounds, the effect of aldosterone receptor antagonists in combination with endothelin receptor antagonists and/or ECE inhibitors is therefore potentially greater than additive.

Advantages of Combination Therapy

The co-administration of an aldosterone receptor antagonist and an endothelin receptor antagonist and/or ECE inhibitor of the present invention can potentially provide more than an additive benefit. For example, the combination of hypertension-lowering effect of the combination therapy methods described herein can be greater than the hypertension-lowering effect of the monotherapeutic administration of each active agent alone. Where the effect is more than additive, a reduced amount of aldosterone receptor antagonist and/or endothelin receptor antagonist and/or ECE inhibitor is needed for combination therapy relative to monotherapy to achieve the desired result.

Accordingly, the combination therapy methods of this invention also can be used to treat or prevent a pathological condition wherein the combination therapy method results in reduced side effects than observed with the corresponding monotherapy to achieve a similar result. For example, reduction of the dose of aldosterone receptor antagonist, endothelin receptor antagonist, and/or ECE inhibitor in the present combination therapy below the conventional monotherapeutic dose can minimize, or even eliminate, the side-effect profile that may be associated with monotherapeutic administration of the drug. In addition, combination therapy methods permit treatment or prevention of a pathological condition to be “fine-tuned” to treat the specific condition of a patient. Thus, by adjusting the dose of the aldosterone receptor antagonist, endothelin receptor antagonist, and/or ECE inhibitor, each compound is provided in a dose that matches the aldosterone, endothelin, and endothelin converting enzyme levels of an individual that need to be inhibited.

Other benefits of the present combination therapy may include, but are not limited to, the use of a selected group of aldosterone receptor antagonists, endothelin receptor antagonists, or ECE inhibitors that provide a relatively quick onset of therapeutic effect and a relatively long duration of action. For example, a single dose of one of the selected antagonists or inhibitors may stay associated with the aldosterone or endothelin receptors for a longer period of time than if provided to a patient on a monotherapeutic basis.

Aldosterone Receptor Antagonists

The term “aldosterone receptor antagonist” denotes a compound capable of binding to an aldosterone receptor, as a competitive inhibitor of the action of aldosterone itself at the receptor site, so as to modulate the receptor-mediated activity of aldosterone.

The aldosterone receptor antagonists used in the methods of the present invention generally are spirolactone-type steroidal compounds. The term “spirolactone-type” is intended to characterize a structure comprising a lactone moiety attached to a steroid nucleus, typically at the steroid “D” ring, through a Spiro bond configuration. A subclass of spirolactone-type aldosterone receptor antagonist compounds consists of epoxy-steroidal aldosterone receptor antagonist compounds such as eplerenone. Another subclass of spirolactone-type antagonist compounds consists of non-epoxy-steroidal aldosterone receptor antagonist compounds such as spironolactone.

The epoxy-steroidal aldosterone receptor antagonist compounds used in the method of the present invention generally have a steroidal nucleus substituted with an epoxy-type moiety. The term “epoxy-type” moiety is intended to embrace any moiety characterized by having an oxygen atom as a bridge between two carbon atoms, examples of which include the following moieties:

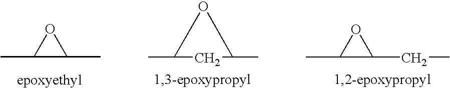

The term “steroidal,” as used in the phrase “epoxy-steroidal,” denotes a nucleus provided by a cyclopenteno-phenanthrene moiety, having the conventional “A,” “B,” “C,” and “D” rings. The epoxy-type moiety may be attached to the cyclopentenophenanthrene nucleus at any attachable or substitutable positions, that is, fused to one of the rings of the steroidal nucleus or the moiety may be substituted on a ring member of the ring system. The phrase “epoxy-steroidal” is intended to embrace a steroidal nucleus having one or a plurality of epoxy-type moieties attached thereto.

Epoxy-steroidal aldosterone receptor antagonists suitable for use in the present methods include a family of compounds having an epoxy moiety fused to the “C” ring of the steroidal nucleus. Examples include 20-spiroxane compounds characterized by the presence of a 9α,11α-substituted epoxy moiety. Compounds 1 through 11, below, are illustrative 9α,11α-epoxy-steroidal compounds that may be used in the present methods. A particular benefit of using epoxy-steroidal aldosterone receptor antagonists, as exemplified by eplerenone, is the high selectivity of this group of aldosterone receptor antagonists for the mineralocorticoid receptor. The superior selectivity of eplerenone results in a reduction in side effects, that can be caused by aldosterone receptor antagonists that exhibit non-selective binding to non-mineralocorticoid receptors, such as androgen or progesterone receptors.

These epoxy steroids may be prepared by procedures described in Grob et al., U.S. Pat. No. 4,559,332. Additional processes for the preparation of 9,11-epoxy steroidal compounds and their salts are disclosed in Ng et al., WO97/21720 and Ng et al., WO98/25948.

TABLE 1
9,11-Epoxy-Steroidal Aldosterone Receptor Antagonists

| Compound # | Structure | Name |
|---|---|---|
| A-1 | 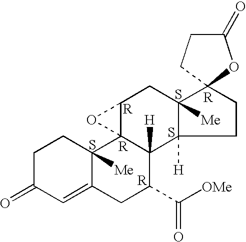 | Pregn-4-ene-7,21-dicarboxylic acid, 9,11-epoxy-17-hy- droxy-3-oxo, γ-lactone, methyl ester, (7α, 11α, 17β)- |
| A-2 | 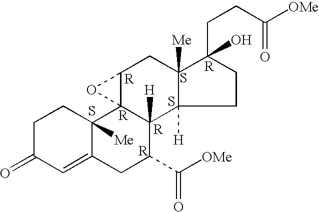 | Pregn-4-ene-7,21-dicarboxylic acid, 9,11-epoxy-17-hy- droxy-3-oxo-, dimethyl ester, (7α, 11α, 17β)- |
| A-3 | 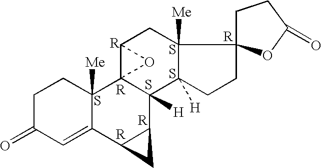 | 3′H-cyclopropa[6,7]pregna-4,6-diene-21-carboxylic acid, 9,11-epoxy-6,7-dihydro-17-hydroxy-3-oxo-, γ-lactone, (6β, 7β, 11α, 17β)- |
| A-4 | 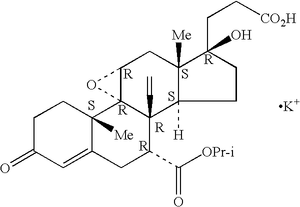 | Pregn-4-ene-7,21-dicarboxylic acid, 9,11-epoxy-17-hy- droxy-3-oxo-, 7-(1-methylethyl)ester, monopotassium salt, (7α, 11α, 17β)- |

Of particular interest is the compound eplerenone (also known as epoxymexrenone). Eplerenone is an aldosterone receptor antagonist and has a higher specificity for aldosterone receptors than does, for example, spironolactone. Selection of eplerenone as the aldosterone receptor antagonist in the present method would be beneficial to reduce certain side-effects such as gynecomastia that occur with use of aldosterone receptor antagonists having less specificity.

Non-epoxy-steroidal aldosterone receptor antagonists suitable for use in the present methods include a family of spirolactone-type compounds defined by Formula I:

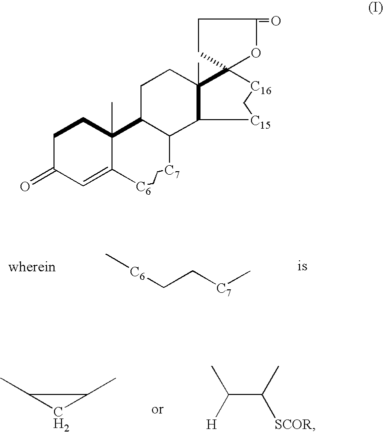

wherein R is lower alkyl of up to 5 carbon atoms, and

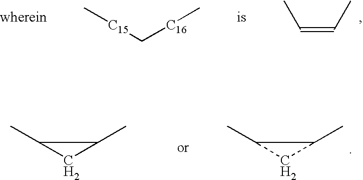

Lower alkyl residues include branched and unbranched groups, for example, methyl, ethyl and n-propyl.

Specific compounds of interest within Formula I are the following:

7α-acetylthio-3-oxo-4,15-androstadiene-[17(β-1′)-spiro-5′]perhydrofuran-2′-one;

3-oxo-7α-propionylthio-4,15-androstadiene-[17((β-1′)-spiro-5′]perhydrofuran-2′-one;

6β,7β-methylene-3-oxo4,15-androstadiene-[17((β-1′)-spiro-5′]perhydrofuran-2′-one;

15α,16α-methylene-3-oxo-4,7α-propionylthio-4-androstene[17(β-1′)-spiro-5′]perhydrofuran-2′-one;

6β,7β,15α,16α-dimethylene-3-oxo-4-androstene[17(β-1′)-spiro-5′]-perhydrofuran-2′-one;

7α-acetylthio-15β,16β-Methylene-3-oxo-4-androstene-[17(β-1′)-spiro-5′]perhydrofuran-2′-one;

15β,16β-methylene-3-oxo-7β-propionylthio-4-androstene-[17(β-1′)-spiro-5′]perhydrofuran-2′-one; and

6β,7β,15β,16β-dimethylene-3-oxo-4-androstene-[17(β-1′)-spiro-5′]perhydrofuran-2′-one.

Methods to make compounds of Formula I are described in U.S. Pat. No. 4,129,564 to Wiechart et al. issued on 12 Dec. 1978.

Another family of non-epoxy-steroidal compounds of interest is defined by Formula II:

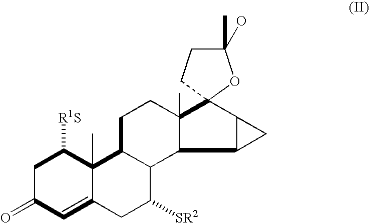

wherein R¹ is C₁₋₃-alkyl or C₁₋₃ acyl and R² is H or C₁₋₃-alkyl.

Specific compounds of interest within Formula II are the following:

1α-acetylthio-15β,16β-methylene-7α-methylthio-3-oxo-17α-pregn-4-ene-21,17-carbolactone; and

15β,16β-methylene-1α,7α-dimethylthio-3-oxo-17α-pregn-4-ene-21,17-carbolactone.

Methods to make the compounds of Formula II are described in U.S. Pat. No. 4,789,668 to Nickisch et al. which issued 6 Dec. 1988.

Yet another family of non-epoxy-steroidal compounds of interest is defined by a structure of Formula III:

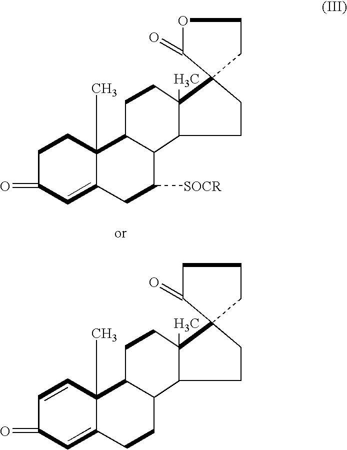

wherein R is lower alkyl, examples of which include lower alkyl groups of methyl, ethyl, propyl and butyl. Specific compounds of interest include:

3β,21-dihydroxy-17α-pregna-5,15-diene-17-carboxylic acid γ-lactone;

3β,21-dihydroxy-17α-pregna-5,15-diene-17-carboxylic acid γ-lactone 3-acetate;

3β,21-dihydroxy-17α-pregn-5-ene-17-carboxylic acid γ-lactone;

3β,21-dihydroxy-17α-pregn-5-ene-17-carboxylic acid γ-lactone 3-acetate;

21-hydroxy-3-oxo-17α-pregn-4-ene-17-carboxylic acid γ-lactone;

21-hydroxy-3-oxo-17α-pregna-4,6-diene-17-carboxylic acid γ-lactone;

21-hydroxy-3-oxo-17α-pregna-1,4-diene-17-carboxylic acid γ-lactone;

7α-acylthio-21-hydroxy-3-oxo-17α-pregn-4-ene-17-carboxylic acid γ-lactone; and

7α-acetylthio-21-hydroxy-3-oxo-17α-pregn-4-ene-17-carboxylic acid γ-lactone.

Methods to make the compounds of Formula III are described in U.S. Pat. No. 3,257,390 to Patchett which issued 21 Jun. 1966.

Still another family of non-epoxy-steroidal compounds of interest is represented by Formula IV:

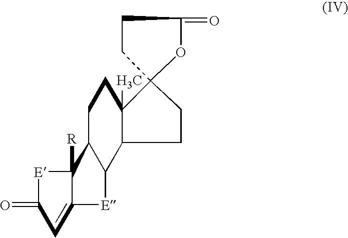

wherein E′ is selected from the group consisting of ethylene, vinylene and (lower alkanoyl)thioethylene radicals, E″ is selected from the group consisting of ethylene, vinylene, (lower alkanoyl)thioethylene and (lower alkanoyl)thiopropylene radicals; R is a methyl radical except when E′ and E″ are ethylene and (lower alkanoyl)thioethylene radicals, respectively, in which case R is selected from the group consisting of hydrogen and methyl radicals; and the selection of E′ and E″ is such that at least one (lower alkanoyl)thio radical is present.

One family of non-epoxy-steroidal compounds within Formula IV is represented by Formula V:

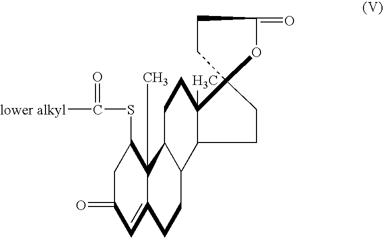

Another compound of Formula V is 1-acetylthio-17α-(2-carboxyethyl)-17β-hydroxy-androst-4-en-3-one lactone.

Another family of non-epoxy-steroidal compounds within Formula IV is represented by Formula VI:

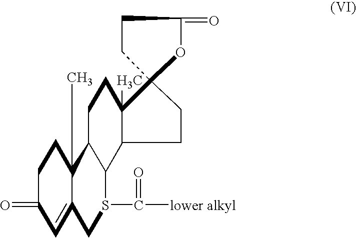

Exemplary compounds within Formula VI include the following:

7α-acetylthio-17α-(2-carboxyethyl)-17β-hydroxy-androst-4-en-3-one lactone;

7β-acetylthio-17α-(2-carboxyethyl)-17β-hydroxy-androst-4-en-3-one lactone;

1α,7α-diacetylthio-17α-(2-carboxyethyl)-17β-hydroxy-androsta-4,6-dien-3-one lactone;

7α-acetylthio-17α-(2-carboxyethyl)-17β-hydroxy-androsta-1,4-dien-3-one lactone;

7α-acetylthio-17α-(2-carboxyethyl)-17β-hydroxy-19-norandrost-4-en-3-one lactone; and

7α-acetylthio-17α-(2-carboxyethyl)-17β-hydroxy-6α-methylandrost-4-en-3-one lactone;

In Formulae IV-VI, the term “alkyl” is intended to embrace linear and branched alkyl radicals containing one to about eight carbons. The term “(lower alkanoyl)thio” embraces radicals of the formula lower alkyl

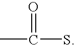

Of particular interest is the compound spironolactone having the following structure and formal name:

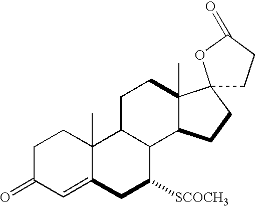

“spironolactone”: 17-hydroxy-7α-mercapto-3-oxo-17α-pregn-4-ene-21-carboxylic acid y-lactone acetate.

Methods to make compounds of Formulae IV-VI are described in U.S. Pat. No. 3,013,012 to Cella et al. which issued 12 Dec. 1961. Spironolactone is sold by G. D. Searle & Co., Skokie, Ill., under the trademark “ALDACTONE”, in tablet dosage form at doses of 25 mg, 50 mg and 100 mg per tablet.

Another family of steroidal aldosterone receptor antagonists is exemplified by drospirenone, [6R-(6α, 7α, 8β, 9α, 10β, 13β, 14α, 15α, 16α, 17β)]-1, 3′, 4′, 6, 7, 8, 9, 10, 11, 12, 13, 14, 15, 16, 20, 21-hex adecahydro-10, 13-dimethylspiro[17H-dicyclopropa[6,7:15,16]cyclopenta[a]phenanthrene-17,2′(5′H)-furan]-3,5′(2H)-dione, CAS registration number 67392-87-4. Methods to make and use drospirenone are described in patent GB 1550568 1979, priority DE 2652761 1976.

Crystalline forms that are easily handled, reproducible in form, easily prepared, stable, and which are non-hygroscopic have been identified for the aldosterone receptor antagonist eplerenone. These include Form H, Form L, various crystalline solvates and amorphous eplerenone. These forms, methods to make these forms, and use of these forms in preparing compositions and medicaments, are disclosed in Barton et al., WO 01/41535 and Barton et al., WO 01/42272.

In one embodiment, form H of eplerenone may be administered in a crystalline form in combination with an endothelin receptor antagonist and/or an ECE inhibitor. In another embodiment, form L of eplerenone may be administered in a crystalline form in combination with an endothelin receptor antagonist and/or an ECE inhibitor. In another embodiment, a mixture of forms H and L may be may be administered in a crystalline form in combination with an endothelin receptor antagonist and/or an ECE inhibitor. In still another embodiment, the amorphous form of eplerenone may be administered in combination with an endothelin receptor antagonist and/or an ECE inhibitor.

Endothelin Receptor Antagonists

Endothelin receptor antagonists, as defined above, encompass a wide range of structures and are useful in the combinations and methods of the present invention. Nonlimiting examples of endothelin receptor antagonists that may be used in the present invention include those endothelin receptor antagonists disclosed in Table 2, including the diastereomers, enantiomers, racemates, salts, esters, tautomers, conjugate acids, and prodrugs of the endothelin receptor antagonists of Table 2. The therapeutic compounds of Table 2 can be used in the present invention in a variety of forms, including acid form, salt form, racemates, enantiomers, zwitterions, and tautomers. The endothelin receptor antagonist references identified in Table 2 are incorporated herein in their entirety.

In one embodiment, a combination therapy comprises administering a first amount of a 9,11-epoxy-steroidal aldosterone receptor antagonist and a second amount of an endothelin receptor antagonist wherein the 9,11-epoxy-steroidal aldosterone receptor antagonist compound is selected from the group of 9,11-epoxy-steroidal aldosterone receptor antagonists disclosed in Table 1, above, and the endothelin receptor antagonist is selected from the group consisting of endothelin receptor antagonists listed below in Table 2. The first amount of the 9,11-epoxy-steroidal aldosterone receptor antagonist and the second amount of the endothelin receptor antagonist together comprise a therapeutically effective amount for the prophylaxis or treatment of a pathological condition.

In another embodiment, a combination therapy comprises administering a first amount of a 9,11-epoxy-steroidal aldosterone receptor antagonist and a second amount of an endothelin receptor antagonist wherein the 9,11-epoxy-steroidal aldosterone receptor antagonist compound is selected from the group of 9,11-epoxy-steroidal aldosterone receptor antagonists disclosed in Table 1, above, and the endothelin receptor antagonist is selected endothelin receptor antagonist compounds other than biphenyl sulfonamide compounds. More preferably, from the group consisting of endothelin receptor antagonists other than biphenyl sulfonamide compounds that are listed below in Table 2. The first amount of the 9,11-epoxy-steroidal aldosterone receptor antagonist and the second amount of the endothelin receptor antagonist together comprise a therapeutically effective amount for the prophylaxis or treatment of a pathological condition.

In another embodiment, the combination therapy comprises administering a first amount of spironolactone and a second amount of an endothelin receptor antagonist, wherein (a) the endothelin receptor antagonist is selected from the group consisting of endothelin receptor antagonists listed below in Table 2, and (b) the first amount of spironolactone and the second amount of the endothelin receptor antagonist together comprise a therapeutically effective amount for the prophylaxis or treatment of a pathological condition.

In another embodiment, the combination therapy comprises administering a first amount of eplerenone and and a second amount of an endothelin receptor antagonist, wherein (a) the endothelin receptor antagonist is selected from the group consisting of endothelin receptor antagonists listed below in Table 2, and (b) the first amount of eplerenone and the second amount of the endothelin receptor antagonist together comprise a therapeutically effective amount for the prophylaxis or treatment of a pathological condition.

TABLE 2
Endothelin Receptor Antagonists

| COMPOUNDS AND COMPOUND | |
|---|---|
| CLASSES | REFERENCE/MANUFACTURER |
| bosentan | U.S. Pat. No. 5,883,254; (CAS No. |
| | 157212-55-0); Roche Holding |
| | AG, Actelion, Genentech |
| sitaxsentan | U.S. Pat. No. 5,594,021; (CAS No. |
| | 184036-34-8); ICOS-Texas |
| | Biotechnology, L.P. |
| darusentan | WO 99/16446; (CAS No. 221176- |
| BMS-187308 | Bristol-Meyers Squibb; Clin. |
| | Cardiol. Vol. 23, Oct. |
| | 2000. |
| BMS-193884 | Bristol-Meyers Squibb; |
| | Pharmacotherapy 22(1): 54-65, |
| | 2002. |
| BMS-20794 | Bristol-Meyers Squibb; |
| | Pharmacotherapy 22(1): 54-65, |
| | 2002. |
| BSF-208075; ambrisentan | Abbott Laboratories, Myogen, |
| | Inc. |
| CGS-27830 | Novartis; Pharmacotherapy |
| | 22(1): 54-65, 2002. |
| IRL-3630 | Novartis; Pharmacotherapy |
| | 22(1): 54-65, 2002. |
| IRL-1038 | |
| enrasentan | SmithKline Beecham |
| FR-139317 | Fujisawa Pharmaceutical Co, |
| | Ltd.; Pharmacotherapy |
| | 22(1): 54-65, 2002. |
| J-104121 | Merck/Banyu; Pharmacotherapy |
| | 22(1): 54-65, 2002. |
| J-104132 | Merck/Banyu; Pharmacotherapy |
| | 22(1): 54-65, 2002. |
| EMD-94246 | Merck; Pharmacotherapy |
| | 22(1): 54-65, 2002. |
| L-744453 | Merck; Pharmacotherapy |
| | 22(1): 54-65, 2002. |
| L-749329 | Merck; Pharmacotherapy |
| | 22(1): 54-65, 2002. |
| L-753037 | Merck; Pharmacotherapy |
| | 22(1): 54-65, 2002. |
| L-754142 | Merck; Pharmacotherapy |
| | 22(1): 54-65, 2002. |
| LU135252 | Knoll AG; Pharmacotherapy |
| | 22(1): 54-65, 2002. |
| LU208075 | Knoll AG; Pharmacotherapy |
| | 22(1): 54-65, 2002. |
| LU302146 | Knoll AG; Pharmacotherapy |
| | 22(1): 54-65, 2002. |
| LU224332 | Knoll AG; Pharmacotherapy |
| | 22(1): 54-65, 2002. |
| LU302872 | Knoll AG; Pharmacotherapy |
| | 22(1): 54-65, 2002. |
| PD-142893 | Parke-Davis; Pharmacotherapy |
| | 22(1): 54-65, 2002. |
| PD-145065 | Parke-Davis; Pharmacotherapy |
| | 22(1): 54-65, 2002. |
| PD-147953 | Parke-Davis; Pharmacotherapy |
| | 22(1): 54-65, 2002. |
| PD-156123 | WO95/05376 |
| RO46-2005 | Hoffmann-La Roche; |
| | Pharmacotherapy 22(1): 54-65, |
| | 2002. |
| RO47-0203 | Hoffmann-La Roche; |
| | Pharmacotherapy 22(1): 54-65, |
| | 2002. |
| RO 48-5695 | Hoffmann-La Roche; |
| | Pharmacotherapy 22(1): 54-65, |
| | 2002. |
| RO 61-1790 | Hoffmann-La Roche; |
| | Pharmacotherapy 22(1): 54-65, |
| | 2002. |
| RO-61-0612 | Roche; Clin. Cardiol. Vol. |
| | 23, Oct. 2000. |
| SB-209670 | SmithKline Beecham; |
| | Pharmacotherapy 22(1): 54-65, |
| | 2002. |
| SB-217242 | SmithKline Beecham; |
| | Pharmacotherapy 22(1): 54-65, |
| | 2002. |
| SB-234551 | SmithKline Beecham; |
| | Pharmacotherapy 22(1): 54-65, |
| | 2002. |
| SB-247083 | SmithKline Beecham; |
| | Pharmacotherapy 22(1): 54-65, |
| | 2002. |
| TA-0115 | Tanabe Seiyaku Co.; |
| | Pharmacotherapy 22(1): 54-65, |
| | 2002. |
| TA-0201 | Tanabe Seiyaku Co.; |
| | Pharmacotherapy 22(1): 54-65, |
| | 2002. |
| TBC11251 | Texas Biotechnology Co.; |
| | Pharmacotherapy 22(1): 54-65, |
| | 2002. |
| TBC-3711 | Texas Biotechnology Co. |
| TBC-11251 | Texas Biotechnology Co.; Clin. |
| | Cardio. Vol. 23, Oct. 2000. |
| ZD 1611 | Zeneca Group plc.; |
| | Pharmacotherapy 22(1): 54-65, |
| | 2002. |
| Sulphisoxazole (4-Amino-N- | (CAS No. 127-69-5); Biochem. |
| (3,4-dimethyl-5-isoxazolyl) | Biophys. Res. Comm. 201 228 |
| benzenesulfonamide) | |
| Sulfonamide derivatives | WO 01/049685; Texas |
| | Biotechnology Corp. |
| 3-Sulfamoyl-pyrazole | EP 1072597; Pfizer Ltd. |
| derivatives | |
| Biphenyl isoxazole | U.S. Pat. No. 6,313,308, WO 00/056685; |
| sulfonamide compounds | Bristol Myers Squibb Co. |
| 4-Heterocyclyl-sulfonamidyl- | WO 00/052007; Hoffmann LaRoche |
| 6-methoxy-5-(2- | & Co. |
| methoxyphenoxy)-2-pyridyl- | |
| pyrimidine derivatives and | |
| their salts | |
| 3-acylamino-propionic acid | EP 1140867; BASF AG |
| and 3-sulfonylamino-propionic | |
| acid derivatives | |
| Phenylsulfonamide derivatives | U.S. Pat. No. 6,107,320; Bristol-Myers |
| and their salts | Squibb Co. |
| Pyrrole derivatives and their | JP 2000063354; Sumitomo |
| acid and alkali salts | Seiyaku, KK |
| Furanone and thiophenone | U.S. Pat. No. 6,017,916; Warner Lambert |
| derivatives | Co. |
| Pyrimidyl sulfonamide | EP 959072; Tanabe Seiyaku Co. |
| derivatives | |
| Pyrimidyl sulfonamide | EP 959073; Tanabe Seiyaku Co. |
| derivatives | |
| Benzothiazine derivatives, | GB 2337048; Warner Lambert Co. |
| their acid addition and base | |
| salts | |
| Phenyl isoxazole sulfonamide | U.S. Pat. No. 5,939,446; Bristol-Myers |
| derivatives, their | Squibb Co. |
| enantiomers, diastereomers | |
| and salts | |
| 5-benzodioxolyl- | EP 1049691, Banyu Pharm Co. |
| cyclopentenopyridine | Ltd. |
| derivatives, including 5- | |
| (2,2-Difluoro-1,3- | |
| benzodioxol-5-yl) | |
| cyclopentenopyridine | |
| derivatives and (5S, 6R, 7R)- | |
| 6-carboxy-5-(2,2-difluoro- | |
| 1,3-benzodioxol-5-yl)-7-(2- | |
| (3-hydroxy-2-methylpropyl)-4- | |
| methoxyphenyl)-2-N- | |
| isopropylaminocyclopentene (1, | |
| 2-b)pyridine | |
| Amino acid derivatives and | U.S. Pat. No. 5,922,681; Warner Lambert |
| their salts including (R-(R*, | Co. |
| S*))-gamma-((3-(1H-indol-3- | |
| yl)-2-methyl-1-oxo-2- | |
| (((tricyclo(3.3.1.13,7)dec-2- | |
| yloxy)carbonyl)amino) | |
| propyl)amino)- | |
| benzenepentanoic acid | |
| 15-ketoprostaglandin E | U.S. Pat. No. 6,197,821, EP 978284; R- |
| compound provided that it | Tech Ueno Ltd. |
| does not contain an alpha | |
| bonded 8C or more backbone, | |
| including 13,14-dihydro-15- | |
| keto-16,16-difluoro-18S- | |
| methylprostaglandin E1 | |
| Pyridyl-thiazole derivatives | U.S. Pat. No. 5,891,892; Warner Lambert |
| | Co. |
| Pyrrolidine and piperidine | U.S. Pat. No. 6,162,927, EP 1003740; |
| derivatives, their analogues | Abbott Laboratories |
| and salts | |
| Pyrrolidine carboxylic acid | U.S. Pat. No. 6,124,341, EP 991620; |
| derivatives, their salts and | Abbott Laboratories |
| stereoisomers | |
| Biphenyl derivatives of | U.S. Pat. No. 5,846,985; Bristol-Myers |
| formula (I), their | Squibb Co. |
| enantiomers, diastereomers, | |
| and salts | |
| Compound S-19777 of formula | JP 10306087; Sankyo Co. Ltd. |
| (I) | |
| Sulphonamide derivatives of | JP 10194972; Tanabe Seiyaku |
| formula (I) and their salts | Co. |
| Prostanoic acid derivative | U.S. Pat. No. 6,242,485, EP 857718; R- |
| with an alpha-chain of at | Tech Ueno Ltd. |
| least 8 skeletal C | |
| Aminoalkoxy or sulpho-alkoxy | U.S. Pat. No. 6,133,263, WO 9737986; |
| furan-2-ones or thiophen-2- | Warner Lambert Co. |
| ones, all of formula (I), and | |
| their salts | |
| Aminoalkoxy 5-hydroxyfuran-2- | U.S. Pat. No. 6,297,274, WO 9737985, |
| ones, their aminoalkylamino | Warner Lambert Co. |
| and alkyl-sulphonic acid | |
| analogues, all of formula | |
| (I), their tautomeric open- | |
| chain keto-acid forms, and | |
| their salts | |
| Pyrrolidine derivatives | EP 888340; Abbott Laboratories |
| Phenylalanine derivatives of | U.S. Pat. No. 5,658,943; Warner Lambert |
| formula (I) | Co. |
| N-isoxazolyl- | U.S. Pat. No. 6,271,248, U.S. Pat. No. 6,080,774, EP |
| biphenylsulphonamide | 768305; Bristol-Myers Squibb |
| derivatives of formula (I) | Co. |
| and their salts, including N- | |
| (3,4-di methyl-5-isoxazolyl)- | |
| 2′-(hydroxymethyl) (1,1′-bi | |
| phenyl)-2-sulphonamide | |
| 3-Aryl (or cycloalkyl) 5H- | U.S. Pat. No. 5,998,468, WO 9708169; |
| furan-2-ones of formula (I) | Warner Lambert Co. |
| and their salts, solvates, | |
| and hydrates | |
| N-Isoxazolyl-4′- | U.S. Pat. No. 5,612,359; Bristol-Myers |
| heterocyclyl(alkyl)-1,1′- | Squibb Co. |
| biphenyl-2-sulphonamides of | |
| formula (I) and their | |
| enantiomers, diastereomers | |
| and salts | |
| Thieno(2,3-d) pyrimidine | U.S. Pat. No. 6,140,325, EP 846119; |
| derivatives (I) contg. a | Takeda Chem. Ind. Ltd. |
| carboxyl gp. or ester and a | |
| gp. other than carboxyl | |
| which is capable of forming | |
| an anion or a gp. | |
| convertible to it | |
| 2(5H)-Furanone derivatives of | U.S. Pat. No. 5,922,759, U.S. Pat. No. 6,017,951, WO |
| formula (I) and their salts | 9702265; Warner Lambert Co. |
| Heterocyclic pyridine | U.S. Pat. No. 6,258,817, U.S. Pat. No. 6,060,475, U.S. Pat. No. |
| sulphonamide derivatives of | 5866568, EP 832082; ZENECA |
| formula (I) and their N | LTD. |
| oxides, salts and prodrugs | |
| Dihydropyridine carboxylic | U.S. Pat. No. 5,576,439; Ciba Geigy Corp. |
| acid anhydride derivatives of | |
| formula (I) and their salts | |
| N-pyrimidinyl-sulphonamide | U.S. Pat. No. 5,739,333, EP 743307; |
| derivatives of formula (I) | Tanabe Seiyaku Co. |
| and their salts | |
| Aroylamidoacyl di-C-substd. | U.S. Pat. No. 5,977,075, EP 821670, |
| glycine derivatives of | Novartis AG |
| formula (I) and their salts | |
| Benzothiazine dioxides of | U.S. Pat. No. 5,599,811, EP 811001; |
| formula (I) and their salts | Warner Lambert Co. |
| N-Isoxazolyl-4′-substd.-1,1′- | U.S. Pat. No. 5,760,038, EP 725067; |
| biphenyl-2-sulphonamide | Bristol-Myers Squibb Co. |
| derivatives of formula (I) | |
| and their enantiomers, | |
| diastereomers and salts | |
| 4-Oxo-2-butenoic acid | WO 9623773, JP 8523414; Banyu |
| derivatives of formula (I) | Pharm Co. Ltd. |
| and 3-hydroxy-2(5H)-furanone | |
| derivatives of formula (II), | |
| and their salts | |
| Aza-aminoacids of formula (I) | ZA 9501743; Abbott |
| | Laboratories |
| Sulphonamides of formula (I) | U.S. Pat. No. 6,004,965, EP 799209; |
| and their salts | Hoffmann La Roche & Co. |
| Aryl compounds of formula | U.S. Pat. No. 6,207,686, EP 792265; |
| (I) and their salts | Fujisawa Pharm Co. Ltd. |
| Phenoxyphenylacetic acid | U.S. Pat. No. 5,559,135, WO 9608487; |
| derivatives and analogues of | Merck & Co. Inc. |
| formula (I) and their salts | |
| 3- (and 5-) Benzene- | U.S. Pat. No. 5,514,696; Bristol-Myers |
| sulphonamido-isoxazole | Squibb Co. |
| derivatives of formula (I) | |
| and their salts | |
| Endothelin antagonists of | ZA 9500892; Abbott |
| formula (I) and their salts, | Laboratories |
| esters and prodrugs | |
| Phenoxyphenylacetic acid | U.S. Pat. No. 5,538,991, WO 9608486; |
| derivatives of formula (I) | Merck & Co. Inc. |
| and their salts | |
| N-Isoxazolyl-4;- | EP 702012; Bristol-Myers |
| heteroar(alk)yl-biphenyl-2- | Squibb Co. |
| sulphonamide derivatives of | |
| formula (I) and their | |
| enantiomers, diastereomers | |
| and salts | |
| Pyrrolidine and piperidine | U.S. Pat. No. 5,622,971, U.S. Pat. No. 5,731,434, U.S. Pat. No. |
| derivatives of formula (I) | 5,767,144, EP 776324; Abbott |
| and their salts | Laboratories |
| Peptide derivatives of | U.S. Pat. No. 5,550,110, EP 767801; |
| formula (I) and their salts | Warner Lambert Co. |
| Porphyrins of formula (I) or | JP 7330601; Kowa Co. Ltd. |
| their metal complexes or | |
| salts | |
| Triazine or pyrimidine | U.S. Pat. No. 5,840,722, EP 752854; BASF |
| derivatives of formula (I) | AG |
| Bicyclic piperazinone | DE 4341663; BASF AG |
| derivatives of formula (I) | |
| and their salts | |
| Benzenesulphonamide | U.S. Pat. No. 5,728,706, EP 658548; |
| derivatives of formula (I), | Tanabe Seiyaku Co. |
| and their salts, including 4- | |
| tert-butyl-N-(5-(4- | |
| methylphenyl)-6-(2-(5-(3- | |
| thienyl)pyrimidin-2- | |
| yloxy)ethoxy)pyrimidin-4-yl)- | |
| benzenesulphonamide | |
| RES-1214 of formula (I) | JP 7133254; Kyowa Hakko Kogyo |
| Bicyclic pyrimidine or 1,4- | U.S. Pat. No. 5,693,637, EP 733052, EP |
| diazepine derivatives of | 733052; BASF AG., Hoechst AG. |
| formula (I) and their acid | |
| addn. salts | |
| 5,11-Dihydro-11-oxo- | U.S. Pat. No. 5,420,123; Bristol-Myers |
| dibenzo(b,e) diazepine | Squibb Co. |
| derivatives of formula (I) | |
| Diaryl- and aryloxy compounds | U.S. Pat. No. 6,211,234, EP 728128; Rhone |
| of formula (I), their salts, | Poulenc Rorer Ltd. |
| N-oxides and prodrugs | |
| Non-peptidic compounds | U.S. Pat. No. 5,492,917, WO 9508989; |
| incorporating a cyclobutane | Merck & Co. Inc. |
| ring of formula (I) and their | |
| salts | |
| Amino acid derivatives of | WO 9508550; Abbott |
| formula (I) and their salts | Laboratories |
| Substituted 2(5H) furanone, | EP 714391; Warner Lambert Co. |
| 2(5H) thiophenone and 2(5H) | |
| pyrrolone derivatives of | |
| formula (I) and their salts | |
| Cyclopentene derivatives of | U.S. Pat. No. 5,714,479, EP 714897; Banyu |
| formula (I) and their salts | Pharm Co. Ltd. |
| Cyclopentane derivatives of | WO 9505372; Banyu Pharm Co. |
| formula (I) and their salts | Ltd. |
| Thienopyrimidine deriv. of | EP 640606; Takeda Chem. Ind. |
| formula (I) or one of its | Ltd., Takeda Pharm Ind. Co. |
| salts | Ltd. |
| Heteroaromatic ring-fused | U.S. Pat. No. 5389620, U.S. Pat. No. 5714479, EP |
| cyclopentene derivatives of | 714897; Banyu Pharm Co. Ltd. |
| formula (I), and their salts | |
| Phenalkyl substd. phenyl | U.S. Pat. No. 5,686,478, EP 710235; Merck |
| compounds of formula (I) and | & Co. Inc. |
| their salts | |
| Benzimidazolinone compounds | U.S. Pat. No. 5,391,566, WO 9503044; |
| substd. with | Merck & Co. Inc. |
| phenoxyphenylacetic acid | |
| derivatives of formula (I) | |
| and their salts | |
| Triterpene derivatives of | JP 6345716; Shionogi & Co. |
| formula (I) and their salts | Ltd. |
| N-Acyl-N-(amino- or hydroxy- | U.S. Pat. No. 5,888,972, EP 706532; |
| alkyl)-tripeptide derivatives | Fujisawa Pharm Co. Ltd. |
| of formula (I) and their | |
| salts | |
| Naphthalenesulphonamido- | U.S. Pat. No. 5,378,715; Bristol-Myers |
| isoxazoles of formula (I) and | Squibb Co. |
| their salts | |
| Amino acid phosphonic acid | U.S. Pat. No. 5,481,030, EP 639586; ADIR |
| derivatives of formula (I), | & CIE |
| their enantiomers, | |
| diastereoisomers, epimers and | |
| salts | |
| Endothelin antagonist of | U.S. Pat. No. 5,420,133; Merck & Co Inc |
| formula (I) or its salts | |
| Peptide derivatives for | WO 9419368; Banyu Pharm Co Ltd |
| formula (I) and their salts | |
| Endothelin antagonist of | U.S. Pat. No. 5,374,638; Merck & Co Inc. |
| formula (I) or its salts | |
| Compounds of formula (I), and | U.S. Pat. No. 5,352,800; Merck & Co. Inc. |
| their salts | |
| 1,4-Dihydro-4-quinolinones | U.S. Pat. No. 5,985,894, EP 498721; |
| and related compounds of | Roussel-Uclaf, Hoechst Marion |
| formula (I) and their isomers | Roussel |
| and salts | |
| Cyclic depsipeptide of | GB 2266890; Merck & Co. Inc. |
| formula (I) | |
| Condensed thiadiazole | U.S. Pat. No. 5,550,138, EP 562599; |
| derivatives of formula (I) | Takeda Chem. Ind. Ltd. |
| and their salts | |
| Compounds (I') and their | U.S. Pat. No. 5,550,138, EP 562599; |
| salts | Takeda Chem. Ind. Ltd. |
| Purified cyclic depsipeptide | U.S. Pat. No. 5,240,910; Merck & Co. Inc. |
| endothelin antagonist of | |
| formula (I) | |
| Cochinmycins (IV) and (V) | U.S. Pat. No. 5,240,910; Merck & Co. |
| | Inc. |
| Peptide derivatives (I) or | JP 5194592; Takeda Chem. Ind. |
| their salts | Ltd. |
| Cyclic peptides (I) or salts | JP 5194589; Takeda Chem. Ind. |
| thereof | Ltd. |
| Peptides of formula (I) and | U.S. Pat. No. 5,614,497, EP 552489; |
| their salts | Takeda Chem. Ind. Ltd. |
| Cyclic hexapeptide | EP 552417; Takeda Chem. Ind. |
| derivatives of formula (I) | Ltd. |
| and their salts, including | |
| cyclo-(D-Asp-Trp-Asp-D-Leu- | |
| Leu-D-Trp) (Ia) | |
| Indane and indene derivatives | EP 612244; Smithkline Beecham |
| of formula (I) and their | Corp. |
| salts | |
| Cyclic peptide derivatives of | U.S. Pat. No. 5,616,684, U.S. Pat. No. 5,883,075, EP |
| formula (I) and their salts | 528312; Takeda Chem. Ind. Ltd. |
| Endothelin (ET) analogue | U.S. Pat. No. 5,352,659, EP 499266; |
| peptides of formula (I) and | Takeda Chem. Ind. Ltd. |
| their salts | |
| Cyclic depsipeptides of | EP 496452, U.S. Pat. No. 4,810,692; Merck |
| formula (A) | & Co. Inc. |
| N-((2′-(((4,5-dimethyl-3- | U.S. Pat. No. 6,043,265; Bristol-Myers |
| isoxazolyl)amino)sulfonyl)-4- | Squibb Co. |
| (2-oxazolyl) (1,1′-bi | |
| phenyl)-2-yl)methyl)-N,3,3- | |
| trimethylbutanamide and salts | |
| thereof | |
| N-(4,5-dimethyl-3- | U.S. Pat. No. 6,043,265; Bristol-Myers |
| isoxazolyl)-2′-((3,3- | Squibb Co. |
| dimethyl-2-oxo-1- | |
| pyrrolidinyl)methy 1)-4′-(2- | |
| oxazolyl) (1,1′-biphenyl)-2- | |
| sulfonamide, and salts | |
| thereof. | |
| Substituted biphenyl | U.S. Pat. No. 5,780,473; Abbott |
| sulfonamide compounds of | Laboratories |
| formula (I), their | |
| enantiomers and | |
| diastereomers, and | |
| pharmaceutically acceptable | |
| salts thereof | |
| Compounds of formula (I) and | U.S. Pat. No. 6,162,927; Abbott |
| salts thereof, including | Laboratories |
| intermediates in the process | |
| of preparation | |
| Heterocyclyl-substituted | U.S. Pat. No. 5,780,473 |
| biphenylsulfonamide | |
| Crystalline sodium salt of 2- | WO 2001030767; BASF AG |
| pyrimidinyloxy-3,3- | |
| diphenylpropionic acid | |
| derivative | |
| Phenyl compounds substituted | U.S. Pat. No. 6,124,343; Rhone-Poulenc |
| with heteroaryl (preferably | Rorer Ltd. |
| thienyl methoxy) moieties and | |
| their derivatives | |
| 1,3-benzodioxole compounds | U.S. Pat. No. 6,048,893; Rhone-Poulenc |
| | Rorer Ltd. |
| Biphenyl sulfonamides of | U.S. Pat. No. 1998-91847P, EP 1094816; |
| formula (I) | Bristol-Myers Squibb Co. |
| Compound (I) or its salt | EP 950418; Takeda Chem Ind |
| | Ltd. |
| A carboxylic acid of formula | EP 1014989; Knoll AG |
| (I) or (II), including s- | |
| triazinyl-or pyrimidinyl- | |
| substituted alkanoic acid | |
| derivative | |
| Endothelin antagonist of | AU 739860; Knoll AG |
| formula (I) | |
| N-(3,4-dimethyl-5- | U.S. Pat. No. 5,916,907, U.S. Pat. No. 5,612,359; |
| isoxazolyl)-4′-(2-oxazolyl) | Bristol-Myers Squibb Co. |
| (1,1′-biphenyl)-2- | |
| sulphonamide and its salts | |
| N-((2′-(((4,5-dimethyl-3- | U.S. Pat. No. 5,916,907, U.S. Pat. No. 5,612,359; |
| isoxazolyl) amino)sulphonyl)- | Bristol-Myers Squibb Co. |
| 4-(2-oxazolyl) (1,1′- | |
| biphenyl)-2-yl)methyl)- | |
| N,3,3-trimethyl butanamide | |
| and its salts | |
| Pyrrolidine derivatives of | U.S. Pat. No. 1997-794506, EP 885215; |
| formula (I) and their salts, | Abbott Laboratories |
| including (2R,3R,4S)-2-(3- | |
| fluoro-4-methoxyphenyl)-4- | |
| (1,3-benzodioxol-5-yl)-1-(2- | |
| (N-propyl-N- | |
| pentanesulphonylamino)ethyl)- | |
| pyrrolidine-3-carboxylic acid | |
| Phenoxyphenylacetic acids and | U.S. Pat. No. 5,565,485; Merck & Co., |
| derivatives of the general | Inc. |
| structural formula I | |
| Compounds of the formula I, | U.S. Pat. No. 5,641,793; Zeneca Limited |
| namely novel pyridine | |
| derivatives including N-(2- | |
| pyridyl)sulphonamides, and | |
| pharmaceutically-acceptable | |
| salts thereof | |
| N-heterocyclic sulfonamides | U.S. Pat. No. 5,668,137; Zeneca Ltd. |
| of the formula I, their | |
| pharmaceutically-acceptable | |
| salts, and pharmaceutical | |
| compositions containing them | |
| Phenoxyphenylacetic acids and | U.S. Pat. No. 5,668,176; Merck & Co. |
| derivatives of the general | Inc. |
| structural formula I | |
| Compounds of Formula I and | U.S. Pat. No. 5,691,373; Warner-Lambert |
| the pharmacologically | Company |
| acceptable salts thereof, | |
| including 2-benzo- | |
| >1,3!dioxol-5-yl-4-(4- | |
| methoxyphenyl)-4-oxo-3- | |
| (3,4,5-trimethoxybenzyl)-but- | |
| 2-enoic acid | |
| Phenoxyphenylacetic acids and | U.S. Pat. No. 5,767,310; Merck & Co., |
| derivatives of general | Inc. |
| structural formula (I) | |
| N-heterocyclyl sulphonamide | U.S. Pat. No. 5,861,401, U.S. Pat. No. 6,083,951; |
| derivatives and their | Zeneca Limited |
| pharmaceutically acceptable | |
| salts | |
| Heterocyclic compounds of the | U.S. Pat. No. 5,866,568; Zeneca Limited |
| formula I and salts thereof, | |
| including N-heterocyclyl | |
| sulphonamides | |
| Pyrimidines of formula I | U.S. Pat. No. 5,883,254, 6,121,447, |
| | 6,274,734; Hoffmann-La Roche |
| | Inc. |
| Nonpeptide compounds of | U.S. Pat. No. 6,017,916; Warner-Lambert |
| formula I | Company |
| Ketoacid compounds of the | U.S. Pat. No. 6,043,241; Warner-Lambert |
| formula I and | Company |
| pharmaceutically acceptable | |
| salts thereof. | |
| 1,2-diheteroethylene | U.S. Pat. No. 6,136,971; Roche Colorado |
| sulfonamides | Corporation |
| Compound of the formula (I) | U.S. Pat. No. 6,218,427; Shionogi & Co., |
| and salts or hydrates thereof | Ltd. |
| Peptides of the formula (I) | U.S. Pat. No. 6,251,861; Takeda Chemical |
| and their salts | Industries, Ltd. |
| Substituted pyrazin-2-yl- | U.S. Pat. No. 6,258,817; Zeneca Ltd. |
| sulphonamide (-3-pyridyl) | |
| compounds of formula I, | |
| salts, and pharmaceutical | |
| compositions containing them. | |
| 4,5-Dihydro-(1H)- | U.S. Pat. No. 6,291,485; Teikoku Hormone |
| benz(g)indazole-3-carboxylic | Mfg. Co., Ltd. |
| acid derivatives of formula I | |
| and their salts | |
| Nonpeptide endothelin I | U.S. Pat. No. 6,297,274; Warner-Lambert |
| antagonists of formula I | Company |
| Carboxylic acid derivatives | EP 946524; BASF AG |
| of formula (I) and their | |
| salts, enantiomers and | |
| diastereomers | |
| 4′-Heterocyclyl(alkyl)-N- | U.S. Pat. No. 5,846,990; BRISTOL-MYERS |
| isoxazolyl-biphenyl-2-yl | SQUIBB CO |
| sulphonamides of formula | |
| (I), and their enantiomers, | |
| diastereoisomers, and salts | |
| Biphenyl sulfonamides of | WO 200001389; BRISTOL-MYERS |
| formula (I) | SQUIBB CO |
| Endothelin antagonist of | WO 9916444, EP 1019055; KNOLL |
| formula (I) | AG |
| Endothelin antagonist of | DE 19743140; KNOLL AG |
| formula (I) | |
| Pyrrolidine derivatives of | WO 9730045; ABBOTT |
| formula (I) and their salts | Laboratories |
| Canrenoate Potassium | U.S. Pat. No. 5,795,909 |
| Canrenone | U.S. Pat. No. 5,795,909 |
| Dicirenone | U.S. Pat. No. 5,795,909 |
| Mexrenoate Potassium | U.S. Pat. No. 5,795,909 |
| Prorenoate Potassium | U.S. Pat. No. 5,795,909 |
| 4-amino-5-furyl-2-yl-4H- | Chinese Chemical Letters |
| 1,2,4-triazolethiol | (2003), 14(8), 790-793. |
| derivatives | |
| 3-alkylthio-4-arylideneamino- | Chinese Chemical Letters |
| 5-(2-furyl)-1,2,4-triazole | (2003), 14(8), 790-793. |
| derivatives | |
| BMS-346567 | Abstracts of Papers, 226th ACS |
| | National Meeting, New York, |
| | NY, September 7-11, 2003 |
| | (2003), MEDI-316.; Bristol- |
| | Myers Squibb |
| Alkanesulfonamides of formula I | WO2003055863 |
| Benzo-fused heterocycles of | WO 2003013545 |
| formula I | |
| (S*)-(4,6-dimethylpyrimidin- | WO 2003013545 |
| 2-yloxy)-[(5S*)-2-oxo-5- | |
| phenyl-1-(2,4,6- | |
| trifluorobenzyl)-2,3,4,5- | |
| tetrahydro-1H-benzo[e] | |
| [1,4]diazepin-5-yl]acetic | |
| acid | |
| (S*)-(3,5- | WO 2003013545 |
| dimethoxyphenoxy) [(1S*)-1- | |
| phenyl-1,2,3,4- | |
| tetrahydroisoquinolin-1-yl] | |
| acetic acid | |
| N-phenylimidazole derivatives | U.S. Pat. No. 2003004202; U.S. Pat. No. 2003153567; |
| | U.S. Pat. No. 6,620,826 |
| Pyrimidine-sulfamides of | WO 2002053557 |
| formula I | |
| Arylalkylsulfonamides of | WO 2002024665 |
| formulas I and II | |
| Pyrimidino-pyridazines of | U.S. Pat. No. 2002061889; U.S. Pat. No. 6,670,362 |
| formulas I and II | |
| Arylethenesulfonic acid | U.S. Pat. No. 2003220359 |
| pyrimidinylamides of formula I | |
| Mercaptopyrrolidine | U.S. Pat. No. 2002049243; U.S. Pat. No. 6,541,638 |
| carboxamides related | |
| compounds of formula I | |
| (2S,4R)-4-mercapto-1- | U.S. Pat. No. 2002049243; U.S. Pat. No. 6,541,638 |
| (naphthalene-2- | |
| sulfonyl)pyrrolidine-2- | |
| carboxylic acid methyl(o- | |
| totylcarbamoylmethyl)amide | |
| N-aminocarbonyl-β-alanines of | WO 2001090079 |
| formula I | |
| 4-(4-pyrimidinyloxy)-2-butyn- | U.S. Pat. No. 2003087920 |
| 1-ol derivatives of formulas | |
| I and II | |
| Pyrimidinyloxypropionates of | WO 2001005771 |
| formula I | |
| (S)-2-(4-methoxy-5- | WO 2001005771 |
| methylpyrimidin-2-yloxy)-3- | |
| methoxy-3,3-diphenylpropionic | |
| acid | |
| 2-pyrimidinyloxypropanoates | WO 2000073276 |
| and analogs thereof of | |
| formulas I and II | |
| Pyrrolidinecarboxylates of | U.S. Pat. No. 6,124,341 |
| formulas I and II | |
| N-(pyridylpyrimidinyl) | U.S. Pat. No. 6,417,360 |
| heterocyclysulfonamides | |
| 4-(heterocyclylsulfonamido)- | U.S. Pat. No. 6,242,601 |
| 5-(2-methoxyphenoxy)-2-phenyl | |
| derivatives of formula I | |
| Pyridylpyrimidines of formula I | U.S. Pat. No. 6,242,601 |
| Monoargininyl salts | U.S. Pat. No. 6,300359 |
| (E)-3-[1-n-butyl-5-[2-(2- | U.S. Pat. No. 6,300359 |
| carboxyphenyl)methoxy-4- | |
| chlorophenyl]-1H-pyrazol-4- | |
| yl]-2-[(5-methoxy-2,3- | |
| dihydrobenzofuran-6- | |
| yl)methyl]-prop-2-enoic acid | |
| 3-carbamoylalkoxy-2- | U.S. Pat. No. 6,509,341 |
| aryloxypropionates and | |
| analogs thereof of formula I | |
| Indole derivatives of | U.S. Pat. No. 6,017,945; U.S. Pat. No. 6,136,843; U.S. Pat. No. |
| formula I | 6,306,852; U.S. Pat. No. 2001014677; U.S. Pat. No. |
| | 6,384,070 |
| α-hydroxy acid derivatives of | U.S. Pat. No. 6,686,369 |
| formula I | |
| 4-benzodioxolylpyrrolidine-3- | WO 9730046 |
| carboxylates and analogs | |
| thereof of formula I | |
| Isoxazoles and imidazoles of | U.S. Pat. No. 6,030,970; U.S. Pat. No. 6,174,906 |
| formula I | |
| Furan and thiophene | U.S. Pat. No. 6,017,952; U.S. Pat. No. 6,051,599 |
| derivatives of formulas I and II | |
| N-isoxazolylthiophenesulfon- | U.S. Pat. No. 5,490,962; U.S. Pat. No. 5,518,680; U.S. Pat. No. |
| amides and analogs thereof of | 5,594,021; U.S. Pat. No. 5,962,490; U.S. Pat. No. |
| formulas I and II | 6,139,574; U.S. Pat. No. 6,342,610; U.S. Pat. No. |
| | 6,331,637; U.S. Pat. No. 6,514,518; U.S. Pat. No. |
| | 6,632,829 |
| N-isoxazolyl(hetero) | U.S. Pat. No. 5,571,821; U.S. Pat. No. 5,490,962; U.S. Pat. No. |
| arenesulfonamides of formulas | 5,464,853; U.S. Pat. No. 5,514,691; U.S. Pat. No. |
| I and II | 5,518,680; U.S. Pat. No. 5,591,761; U.S. Pat. No. |
| | 5,594,021; U.S. Pat. No. 5962,490; U.S. Pat. No. |
| | 6,030,991; U.S. Pat. No. 6,139,574; U.S. Pat. No. |
| | 6,331,637; U.S. Pat. No. 6,376,523; U.S. Pat. No. |
| | 6,541,498; U.S. Pat. No. 6,514,518; U.S. Pat. No. |
| | 6,613,804 |
| N-(4-pyrimidinyl)sulfonamides | EP 713875 |
| of formula I | |
| Arylimidazolylpropenoates and | U.S. Pat. No. 2003153567; U.S. Pat. No. 6,620,826 |
| related compounds of formula I | |
| (E)-3-[s-butyl-1-[2-[N- | U.S. Pat. No. 2003153567; U.S. Pat. No. 6,620,826 |
| (phenylsulfonyl)]carboxamido- | |
| 4-methoxyphenyl]-1H-imidazol- | |
| 5-yl]-2-[(2-methoxy-4,5- | |
| methylenedioxyphenyl)methyl]- | |
| 2-propenoic acid dipotassium | |
| salt | |
| Pyrimidine and triazine | U.S. Pat. No. 5,932,730; U.S. Pat. No. 6,197,958; U.S. Pat. No. |
| derivatives of formulas I and | 6,600,043 |
| II | |
| Indane and Indene derivatives | U.S. Pat. No. 6,271,399; U.S. Pat. No. 6,087,389; U.S. Pat. No. |
| of formula I | 6,274,737; U.S. Pat. No. 2002002177; U.S. Pat. No. |
| | 6,448,260 |
| Heteroaromatic ring-fused | U.S. Pat. No. 5,389,620; U.S. Pat. No. 5,714,479 |
| cyclopentene derivatives of | |
| formula I | |
| (5RS,6SR,7RS)-6-carboxy-7-(4- | U.S. Pat. No. 5,389,620; U.S. Pat. No. 5,714,479 |
| methoxyphenyl)-5-(3,4- | |
| methylenedioxyphenyl)cyclopenteno | |
| [1,2-b]pyridine | |
| Pyrido[2,3-d]pyrimidinesof | U.S. Pat. No. 5,654,309 |
| formulas I and II | |
| Pyrido[2,3-d]pyrimidine-3- | U.S. Pat. No. 5,654,309 |
| acetic acid of formula II | |
| 4-Heterocyclyl-sulfonamidyl- | WO 200052007 |
| 6-methoxy-5-(2- | |
| methoxyphenoxy)-2-pyridyl- | |
| pyrimidine derivatives of | |
| formula I | |
| Alpha-hydroxy-carboxylic acid | DE 19614533 |
| derivatives of formula I | |
| 2-(4,6-dimethylpyrimidin-2- | DE 19614533 |
| yloxy)-3,3-diphenylbutyric | |
| acid | |
| 2-formylaniline derivatives | WO 2003080643 |
| of formula V | |
| 6a-{3-[2-(3-carboxy- | WO 2003080643 |
| acryloylamino)-5- | |
| hydroxyphenyl]- | |
| acryloyloxymethyl}- | |
| 2,2,6b,9,9,12a-hexamethyl-10- | |
| oxo1,3,4,5,6,6a,7,8,8a,9,9,12 | |
| a,12b,13,14b-octadecahydro- | |
| 2H-picene-4a-carboxylic acid | |
| or its salts | |
| Alkanesulfonamides of | WO 2003055863 |
| formulas I or Ia | |
| ethanesulfonic acid {6-[2-(5- | WO 2003055863 |
| bromo-pyrimidin-2-yloxy)- | |
| ethoxy]-5-para-tolyl- | |
| pyrimidin-4-yl}-amine | |
| N-phenyl imidazole | U.S. Pat. No. 2003004202 |
| derivatives of formula I or | |
| salts thereof | |
| (E)-3-[2-butyl-1-[2-(2- | U.S. Pat. No. 2003004202 |
| carboxyphenyl)methoxy-4- | |
| methoxy]phenyl-1H-imidazol-5- | |
| yl]-2-[(2-methoxy-4,5- | |
| methylenedioxyphenyl)methyl]- | |
| 2-propenoic acid | |
| Benmzofused heterocycle | WO 2003013545 |
| derivatives of formula I and | |
| salts thereof | |

In one embodiment, a combination therapy comprises administering a first amount of an a 9,11-epoxy-steroidal aldosterone receptor antagonist compound and a second amount of an an endothelin receptor antagonist, wherein the 9,11-epoxy-steroidal aldosterone receptor antagonist compound is selected from the group of 9,11-epoxy-steroidal aldosterone receptor antagonists disclosed in Table 1, above, and the endothelin receptor antagonist is selected from the group consisting of endothelin receptor antagonists listed below in Table 3. The first amount of the 9,11-epoxy-steroidal aldosterone receptor antagonist and the second amount of the endothelin receptor antagonist together comprise a therapeutically effective amount for the prophylaxis or treatment of a pathological condition.

In another embodiment, the combination therapy comprises administering a first amount of spironolactone and an second amount of an endothelin receptor antagonist, wherein (a) the endothelin receptor antagonist is selected from the group consisting of endothelin receptor antagonists listed below in Table 3, and (b) the first amount of spironolactone and an second amount of the endothelin receptor antagonist together comprise a therapeutically effective amount for the prophylaxis or treatment of a pathological condition.

In another embodiment, the combination therapy comprises administering a first amount of eplerenone and and an second amount of an endothelin receptor antagonist, wherein (a) the endothelin receptor antagonist is selected from the group consisting of endothelin receptor antagonists listed below in Table 3, and (b) the first amount of eplerenone and an second amount of the endothelin receptor antagonist together comprise a therapeutically effective amount for the prophylaxis or treatment of a pathological condition.

TABLE 3

| | | | Patent/Literature |
|---|---|---|---|
| | | | Reference for |
| Compound | | CAS Registry | Preparation of |
| Number | Common Name | Number | Compound Per Se |
| B-1 | bosentan | 157212-55-0 | U.S. Pat. No. 5,883,254 |
| B-2 | sitaxsentan | 184036-34-8 | U.S. Pat. No. 5,594,021 |
| B-3 | darusentan | 221176-51-8 | WO 09916466 |
| B-4 | tezosentan | | |
| B-5 | enrasentan | | |
| B-6 | tarasentan | | |
| B-7 | atrasentan | | |
| | (ABT-627) | | |
| B-8 | ambrisentan | | |
| | (BSF-208075) | | |
| B-9 | BMS-187308 | | |
| B-10 | BMS-193884 | | |
| B-11 | J-104132 | | |
| B-12 | PD-145065 | | |
| B-13 | RO-61-0612 | | |
| B-14 | SB-209670 | | |
| B-15 | SB-217242 | | |
| B-16 | SB-247083 | | |
| B-17 | TAK-044 | | |
| B-18 | TBC-3711 | | |
| B-19 | TBC-11251 | | |
| B-20 | ZD-1611 | | |

In another embodiment, a combination therapy comprises administering a first amount of a 9,11-epoxy-steroidal aldosterone receptor antagonist compound and a second amount of an endothelin receptor antagonist, wherein the 9,11-epoxy-steroidal aldosterone receptor antagonist compound is selected from the group of 9,11-epoxy-steroidal aldosterone receptor antagonists disclosed in Table 1, above, and the endothelin receptor antagonist is selected from the group consisting of bosentan, sitaxsentan, darusentan, and tezosentan, listed below in Table 4. The first amount of the 9,11-epoxy-steroidal aldosterone receptor antagonist and the second amount of the endothelin receptor antagonist together comprise a therapeutically effective amount for the prophylaxis or treatment of a pathological condition.

In another embodiment, a combination therapy comprises administering a first amount of spironolactone and an second amount of an endothelin receptor antagonist, wherein (a) the endothelin receptor antagonist is selected from the group consisting of bosentan, sitaxsentan, darusentan, and tezosentan listed below in Table 4, and (b) the first amount of spironolactone and an second amount of the endothelin receptor antagonist together comprise a therapeutically effective amount for the prophylaxis or treatment of a pathological condition.

In still another embodiment, a combination therapy comprises administering a first amount of eplerenone and an endothelin receptor antagonist, wherein (a) the endothelin receptor antagonist is selected from the group consisting of bosentan, sitaxsentan, darusentan, and tezosentan listed below in Table 4, and (b) the first amount of eplerenone and the second amount of the endothelin receptor antagonist together comprise a therapeutically effective amount for the prophylaxis or treatment of a pathological condition.

TABLE 4

| | | | Patent/Literature |
|---|---|---|---|
| | | | Reference for |
| Compound | | CAS Registry | Preparation of |
| Number | Common Name | Number | Compound Per Se |
| C-1 | bosentan | 157212-55-0 | U.S. Pat. No. 5,883,254 |
| C-2 | sitaxsentan | 184036-34-8 | U.S. Pat. No. 5,594,021 |
| C-3 | darusentan | 221176-51-8 | WO 09916466 |
| C-4 | tezosentan | | |

Endothelin Converting Enzyme Inhibitors (ECE Inhibitors) ECE inhibitors, as defined above, encompass a wide range of structures and are useful in the combinations and methods of the present invention. Nonlimiting examples of ECE inhibitors that may be used in the present invention include those ECE inhibitors disclosed in Table 5, below, including the diastereomers, enantiomers, racemates, salts, esters, tautomers, conjugate acids, zwitterions, tautomers, and prodrugs thereof. The ECE inhibitor references identified in Table 5 are incorporated herein in their entirety.

In one embodiment, a combination therapy comprises administering a first amount of a 9,11-epoxy-steroidal aldosterone receptor antagonist compound and a second amount of an ECE inhibitor, wherein the 9,11-epoxy-steroidal aldosterone receptor antagonist compound is selected from the group of 9,11-epoxy-steroidal aldosterone receptor antagonists disclosed in Table 1, above, and the ECE inhibitor is selected from the group consisting of ECE inhibitors listed in Table 5 below. The first amount of the 9,11-epoxy-steroidal aldosterone receptor antagonist and the second amount of the ECE inhibitor together comprise a therapeutically effective amount for the prophylaxis or treatment of a pathological condition.

In another embodiment, a combination therapy comprises administering a first amount of spironolactone and a second amount of an ECE inhibitor, wherein (a) the ECE inhibitor is selected from the group consisting of ECE inhibitors listed below in Table 5, and (b) the first amount of spironolactone and the second amount of the ECE inhibitor together comprise a therapeutically effective amount for the prophylaxis or treatment of a pathological condition.

In another embodiment, a combination therapy comprises administering a first amount of eplerenone and a second amount of an ECE inhibitor, wherein (a) the ECE inhibitor is selected from the group consisting of ECE inhibitors listed below in Table 5, and (b) the first amount of eplerenone and the second amount of the ECE inhibitor together comprise a therapeutically effective amount for the prophylaxis or treatment of a pathological condition.

In still another embodiment, a combination therapy comprises administering a first amount of eplerenone and a second amount of an ECE inhibitor, wherein (a) the ECE inhibitor is selected from the group consisting of CGS 26303, phosphoramidon, FR901533, TMC-66, and SM-19712 and (b) the first amount of eplerenone and the second amount of the ECE inhibitor together comprise a therapeutically effective amount for the prophylaxis or treatment of a pathological condition.

TABLE 5
ECE INHIBITORS

| COMPOUNDS AND COMPOUND | |
|---|---|
| CLASSES | REFERENCE |
| CGS 26303 | J Cardiovasc Pharmacol 2000; |
| | 36(Suppl. 1): S342-S345; |
| | Novartis |
| phosphoramidon | CAS No. 36357-77-4; J. |
| | Cardiovasc. Pharmacol., 1998, |
| | 32(1): 12-20. |
| FR901533 | Fujisawa Pharmaceutical Co, |
| | Ltd |
| TMC-66 (Endothelin Converting | The Journal of Antibiotic, |
| Enzyme Inhibitor Produced by | 1999, Vol. 52 No. 7, 607. |
| Streptomyces sp. A5008) | |
| SM-19712 | Jpn. J. Pharmacol. 84 (1), 16- |
| {4-chloro-N-(((4-cyano-3- | 24 (2000) |
| methyl-1-phenyl-1H-pyrazol-5- | |
| yl)amino)carbonyl) | |
| benzenesulfonamide, | |
| monosodium salt} | |
| SLV-306; (3S,2′R)-3-[1-[2′- | Solvay |
| (Ethoxycarbonyl)-4′-phenyl- | |
| butyl]-cyclopentan-1- | |
| carbonylamino]-2,3,4,5- | |
| tetrahydro-2-oxo-1H-1- | |
| benzazepin-1-acetic acid | |
| KC-12615 (active metabolite | Solvay and Albert Szent- |
| of SLV-306); | Gyorgyi Medical University in |
| (3S,2′R)-3-(1-(2′-Carboxy-4′- | Hungary |
| phenyl-butyl)-cyclopentan-1- | |
| carbonyl-amino)-2,3,4,5- | |
| tetrahydro-2-oxo-1H-1- | |
| benzazepin-1-acetic acid. | |
| KC-90095-1-AC | Solvay and Albert Szent- |
| | Gyorgyi Medical University in |
| | Hungary |
| CGS-26303; | Novartis |
| [N-[2-(Biphenyl-4-yl)-1(S)- | |
| (1H-tetrazol-5-yl)ethyl] | |
| amino]methylphosphonic acid | |
| CGS-30440; | Novartis |
| N-[1-[2(S)- | |
| (Acetylsulfanyl)-3- | |
| methylbutyramido]cyclopent- | |
| 1-ylcarbonyl]-4-O-methyl-L- | |
| tyrosine ethyl ester | |
| CGS-31447; | Novartis |
| [1-[N-[2-(Biphenyl-4-yl)- | |
| 1(S)-(1H-tetrazol-5-yl) | |
| ethyl]amino]-2-(1- | |
| naphtyl)ethyl]phosphonic | |
| acid | |
| CGS-26670; 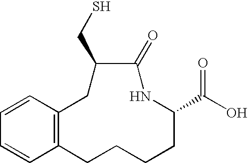 (member of a series of of substituted benzofused macrocyclic lactams) | Novartis |
| Sch-54470; | Schering Plough; |
| N-[1-[Hydroxy[1(R)-[N^{a}- | U.S. Pat. No. 5,476,847 |
| (methylsulfonyl)-1- | |
| lysylamino]-2-phenylethyl] | |
| phosphinylmethyl] | |
| cyclopentylcarbonyl]-1- | |
| tryptophan dilithium salt | |
| Hydrazone derivative | JP 2000302768; Sumitomo |
| Compounds of formula (I) and | Seiyaku Kk |
| formula (II), their prodrugs | |
| and pharmaceutically | |
| acceptable salts | |
| N-((mercapto) (aryl)alkyl)- | EP 888299; Hoechst Marion |
| amide derivatives of formula | Roussel, Roussel-Uclaf |
| (I), its racemates, | |
| enantiomeric or | |
| diastereomeric forms, and | |
| addition salts with mineral | |
| or organic acids or bases, | |
| including (S-(R*,S*))-N-(1- | |
| (mercaptomethyl)-2- | |
| phenylethyl)-alpha- | |
| (((phenylmethoxy) | |
| carbonyl)amino)-1H-indole-3- | |
| propanamide (Ia); 2′-cyano- | |
| alpha-(((1-(mercaptomethyl)- | |
| 2-phenylethyl)amino) | |
| carbonyl)-(1,1′-biphenyl)-4- | |
| propanoic acid (Ib); and (R)- | |
| N-(1-(mercaptomethyl)-2- | |
| phenylethyl)-11-phenoxy- | |
| undecanamide | |
| Endothelin converting enzyme | JP 8208646; Sankyo Co. Ltd. |
| inhibitor B90063 of formula | |
| (I) and its salts | |
| Soya saponin cpd. of formula | JP 7188033; Nisshin Flour |
| (I) and its salts | Milling Co. |
| Anthracycline group compounds | JP 7188034; Nisshin Flour |
| or their salts, including | Milling Co. |
| aclarubicin | |
| Peptide analogues of formula | U.S. Pat. No. 5,338,726, EP 569487; |
| (I)-(III) and their salts, | Abbott Laboratories |
| esters and prodrugs | |
| Aminophosphoric acid | U.S. Pat. No. 5,380,921, EP 623625; Banyu |
| derivitives of formula (I) | Pharm Co. Ltd. |
| and pharmaceutically | |
| acceptable salts, including | |
| N-(N-(1-dibenzyloxy | |
| phosphoryl-3-phenylpropyl)- | |
| L-leucyl)-L-tryptophan benzyl | |
| ester and N-(N-(3-phenyl-1- | |
| phosphonopropyl)-L-leucyl)- | |
| L-tryptophan tripotassium | |
| salt | |
| Phosphonic acid derivs. of | U.S. Pat. No. 5,330,978, U.S. Pat. No. 35,886 |
| formula (I), with endothelin- | (reissue), EP 518299; Takeda |
| converting enzyme inhibiting | Chem Ind. Ltd. |
| activity, and their salts | |
| Metal-containing and metal- | EP 575405; Berlex Lab Inc. |
| free (apoprotein) forms of | |
| compound (I). | |
| Phosphoramide of formula (I) | JP 4041430; Banyu Pharm Co. |
| and its salts | Ltd. |
| Phosphoric acid derivitive of | WO 9201468, JP 3510577; Green |
| formula (I) and its salts | Cross Corp. |
| Ile-Ile-Trp-Phe-Asn-Thr-Pro- | A. Claing et al., 4th Intl. |
| Glu-His-Val-Val-Pro-Tyr-Gly- | Conf. on Endothelin, London, |
| Leu-Gly-Ser-Pro-Arg | 1995 |
| SCH 54470 | |
| B-90063 (Endothelin | |
| Converting Enzyme Inhibitor | |
| Isolated from Blastobacter | |
| sp. SANK 71894) | |
| Benzazepinone-N-acetic acid | U.S. Pat. No. 5,952,327; Solvay |
| derivatives | Pharmaceuticals GmbH |
| The inhibitors of formulae | WO 200149322 |
| (IA) or (IB), and their salts | |
| ECE inhibitor of formula (I), | U.S. Pat. No. 1998-65265P, EP 1073674; |
| (III), and (IIIb) | Novartis AG |
| N-phosphonomethyl substituted | U.S. Pat. No. 5,550,119; CIBA GEIGY CORP |
| derivitives of formula (I) or | |
| its tautomer or salt | |
| ECE inhibitor of formula (I) | WO 9955726, EP 1073674; |
| | NOVARTIS AG |
| 2,5-diamidoindoles | WO 2003028719; Bayer |
| | Aktiengesellschaft, Germany |
| Thiol containing amino acids | U.S. Pat. No. 6,613,782 |
| of formula I | |
| 2-{[(1-(2-mercapto-3- | U.S. Pat. No. 6,613,782 |
| methylbutanoylamino)cyclopent | |
| ane)carbonyl]amino}-3-(2- | |
| methoxybiphenyl-4- | |
| yl)propionic acid | |
| Heteroaryl substituted amino | U.S. Pat. No. 6,689,801 |
| acid thiols of formulas I and | |
| II | |
| N-[3-(3-bromophenyl)-2- | U.S. Pat. No. 6,136,842 |
| (mercaptomethyl)-1- | |
| oxopropyl]-L-tryptophan | |
| Aralkanoamides | U.S. Pat. No. 6,420,341 |
| Quinazolineamines and analogs | U.S. Pat. No. 5,658,902; U.S. Pat. No. 5,773,444 |
| of formula I | |
| Peptideamide derivatives of | U.S. Pat. No. 6,235,717 |
| formula I | |
| N-[5-[(hexahydro-2-oxo-1H- | U.S. Pat. No. 6,235,717 |
| thieno[3,4-d]imidazol-4- | |
| yl)pentanoyl]-L-p- | |
| bromophenylalanyl-L-1- | |
| naphthylalanyl-L-N-[1- | |
| formyl]-2-(1H-indol-3- | |
| yl)ethylamide | |
| Heteroaryl substituted thiol | WO 9955723 |
| derivatives of formula I or | |
| Ia | |
| 2-{[1-2-Mercapto-4-methyl- | WO 9955723 |
| pentanoylamino)- | |
| cyclopentanecarbonyl]-amino}- | |
| 3-[6-(thien-2-yl)-pyridin-3- | |
| yl]-propionic acid | |
| 5-Acylamino-N-phenyl-1H- | DE 10147672 |
| indole-2-carboxamide | |
| derivatives of formula I | |
| 5-((3,3-Dimethylbutanoyl)- | DE 10147672 |
| amino)-1-(2-fluorobenzyl)-N- | |
| phenyl-1H-indole-2- | |
| carboxamide | |

As noted above, the endothelin receptor antagonists and ECE inhibitors useful in the present combination therapy also may include the racemates and stereoisomers, such as diastereomers and enantiomers, of such inhibitors. Such stereoisomers can be prepared and separated using conventional techniques, either by reacting enantiomeric starting materials, or by separating isomers of compounds of the present invention. Isomers may include geometric isomers, for example cis isomers or trans isomers across a double bond. All such isomers are contemplated among the compounds of the present invention. Such isomers may be used in either pure form or in an admixture with those inhibitors described above.

Combinations and Compositions

The present invention is further directed to combinations, including pharmaceutical compositions, comprising one or more aldosterone receptor antagonists and one or more endothelin receptor antagonist and/or ECE inhibitor. In one embodiment, the present invention comprises an aldosterone receptor antagonist, or a pharmaceutically acceptable salt, ester, or prodrug thereof; an endothelin receptor antagonist and/or ECE inhibitor, or a pharmaceutically acceptable salt, ester, conjugate acid, or prodrug thereof; and a pharmaceutically acceptable carrier. The aldosterone receptor antagonist, or a pharmaceutically acceptable salt, ester, or prodrug thereof and the endothelin receptor antagonist and/or ECE inhibitor, or a pharmaceutically acceptable salt, ester, conjugate acid, or prodrug thereof together comprise a therapeutically effective composition for treating pathological conditions.

In one embodiment, the aldosterone receptor antagonists and endothelin receptor antagonist and/or ECE inhibitors used in the preparation of the compositions are as previously set forth above. The combinations and compositions comprising an aldosterone receptor antagonist and an endothelin receptor antagonist and/or ECE inhibitor of the present invention can be administered for the prophylaxis and/or treatment of pathological conditions, as previously set forth, by any means that produce contact of these inhibitors with their site of action in the body.

For the prophylaxis or treatment of the pathological conditions referred to above, the combination administered can comprise the inhibitor compounds per se. Alternatively, pharmaceutically acceptable salts are particularly suitable for medical applications because of their greater aqueous solubility relative to the parent compound.

The combinations of the present invention also can be presented with an acceptable carrier in the form of a pharmaceutical composition. The carrier must be acceptable in the sense of being compatible with the other ingredients of the composition and must not be deleterious to the recipient. The carrier can be a solid or a liquid, or both, and preferably is formulated with the compound as a unit-dose composition, for example, a tablet, which can contain from 0.05% to 95% by weight of the active compounds. Other pharmacologically active substances can also be present, including other compounds useful in the present invention. The pharmaceutical compositions of the invention can be prepared by any of the well-known techniques of pharmacy, such as admixing the components.

The combinations and compositions of the present invention can be administered by any conventional means available for use in conjunction with pharmaceuticals. Oral delivery of the aldosterone receptor antagonist and the endothelin receptor antagonist and/or ECE inhibitor is generally preferred (although the methods of the present invention are still effective, for example, if the endothelin receptor antagonist and/or ECE inhibitor is administered parenterally). The amount of each antagonist and or inhibitor in the combination or composition that is required to achieve the desired biological effect will depend on a number of factors including those discussed below with respect to the treatment regimen.

Oral delivery of the aldosterone receptor antagonist and the endothelin receptor antagonist and/or ECE inhibitor of the present invention can include formulations, as are well known in the art, to provide immediate delivery or prolonged or sustained delivery of the drug to the gastrointestinal tract by any number of mechanisms. Immediate delivery formulations include, but are not limited to, oral solutions, oral suspensions, fast-dissolving tablets or capsules, disintegrating tablets and the like. Prolonged or sustained delivery formulations include, but are not limited to, pH sensitive release from the dosage form based on the changing pH of the small intestine, slow erosion of a tablet or capsule, retention in the stomach based on the physical properties of the formulation, bioadhesion of the dosage form to the mucosal lining of the intestinal tract, or enzymatic release of the active drug from the dosage form. The intended effect is to extend the time period over which the active drug molecule is delivered to the site of action by manipulation of the dosage form. Thus, enteric-coated and enteric-coated controlled release formulations are within the scope of the present invention. Suitable enteric coatings include cellulose acetate phthalate, polyvinylacetate phthalate, hydroxypropylmethyl-cellulose phthalate and anionic polymers of methacrylic acid and methacrylic acid methyl ester.

Pharmaceutical compositions suitable for oral administration can be presented in discrete units, such as capsules, cachets, lozenges, or tablets, each containing a predetermined amount of at least one compound of the present invention; as a powder or granules; as a solution or a suspension in an aqueous or non-aqueous liquid; or as an oil-in-water or water-in-oil emulsion. As indicated, such compositions can be prepared by any suitable method of pharmacy which includes the step of bringing into association the inhibitor(s) and the carrier (which can constitute one or more accessory ingredients). In general, the compositions are prepared by uniformly and intimately admixing the inhibitor(s) with a liquid or finely divided solid carrier, or both, and then, if necessary, shaping the product. For example, a tablet can be prepared by compressing or molding a powder or granules of the inhibitors, optionally with one or more assessory ingredients. Compressed tablets can be prepared by compressing, in a suitable machine, the compound in a free- flowing form, such as a powder or granules optionally mixed with a binder, lubricant, inert diluent and/or surface active/dispersing agent(s). Molded tablets can be made, for example, by molding the powdered compound in a suitable machine.

Liquid dosage forms for oral administration can include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, and sweetening, flavoring, and perfuming agents.

Pharmaceutical compositions suitable for buccal (sub-lingual) administration include lozenges comprising a compound of the present invention in a flavored base, usually sucrose, and acacia or tragacanth, and pastilles comprising the inhibitors in an inert base such as gelatin and glycerin or sucrose and acacia.

In any case, the amount of aldosterone receptor antagonist and endothelin receptor antagonist and/or ECE inhibitor that can be combined with carrier materials to produce a single dosage form to be administered will vary depending upon the host treated and the particular mode of administration. The solid dosage forms for oral administration including capsules, tablets, pills, powders, and granules noted above comprise the inhibitors of the present invention admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Pharmaceutically acceptable carriers encompass all the foregoing and the like. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pa., 1975; Liberman, et al., Eds., Pharmaceutical Dosage Forms, Marcel Decker, New York, N.Y., 1980; and Kibbe, et al., Eds., Handbook of Pharmaceutical Excipients (3_{rd} Ed.), American Pharmaceutical Association, Washington, 1999.

Triple or Multiple Combination Therapy

The present invention is further directed to combinations, including pharmaceutical compositions and the administration of combination therapies thereof comprising an aldosterone receptor antagonist and endothelin receptor antagonist and/or ECE inhibitor and one or more additional active drugs. Such compositions and combination therapies may be utilized for the treatment of pathological conditions such as, for example, hypertension, cardiovascular disease, renal dysfunction, edema, cerebrovascular disease, and insulinopathies and the like. The active drugs co-administered with the aldosterone receptor antagonist and endothelin receptor antagonist and/or ECE inhibitor can include, but are not limited to, for example, drugs selected from the group consisting of renin inhibitors, angiotensin I antagonists, angiotensin II antagonists, angiotensin converting enzyme inhibitors, alpha-adrenergic receptor blockers, beta-adrenergic receptor blockers, calcium channel blockers, neutral endopeptidase inhibitors (such as omapatrilat), vasodilators, cyclooxygenase-1 inhibitors, and diuretics.

Other active drugs that can be co-administered with a aldosterone receptor antagonist and endothelin receptor antagonist and/or ECE inhibitor include, but are not limited to, members of the group consisting of lipid-lowering drugs (including apical sodium bile acid transport inhibitors, cholesterol absorption inhibitors, fibrates, niacin, statins, cholesteryl ester transfer protein inhibitors, and bile acid sequestrants), anti-oxidants (including vitamin E and probucol), and IIb/IIIa antagonists.

Angiotensin-II receptor antagonists that are within the scope of this invention include, but are not limited to: candesartan, which may be prepared as disclosed in U.S. Pat. No. 5,196,444; eprosartan, which may be prepared as disclosed in U.S. Pat. No. 5,185,351; irbesartan, which may be prepared as disclosed in U.S. Pat. No. 5,270,317; losartan, which may be prepared as disclosed in U.S. Pat. No. 5,138,069; and valsartan, which may be prepared as disclosed in U.S. Pat. No. 5,399,578. The disclosures of all such U.S. Patents are incorporated herein by reference.

Angiotensin converting enzyme inhibitors that are within the scope of this invention include, but are not limited to: alacepril, which may be prepared as disclosed in U.S. Pat. No. 4,248,883; benazepril, which may be prepared as disclosed in U.S. Pat. No. 4,410,520; captopril, which may be prepared as disclosed in U.S. Pat. Nos. 4,046,889 and 4,105,776; ceronapril, which may be prepared as disclosed in U.S. Pat. No. 4,452,790; cilazapril, which may be prepared as disclosed in EP 94095 B 1990, delapril, which may be prepared as disclosed in U.S. Pat. No. 4,385,051; enalapril, which may be prepared as disclosed in U.S. Pat. No. 4,374,829; fosinopril, which may be prepared as disclosed in U.S. Pat. No. 4,337,201; imadapril, which may be prepared as disclosed in U.S. Pat. No. 4,508,727; lisinopril, which may be prepared as disclosed in U.S. Pat. No. 4,555,502; moveltopril, which may be prepared as disclosed in Belgian Patent No. 893,553; perindopril, which may be prepared as disclosed in U.S. Pat. No. 4,508,729; quinapril, which may be prepared as disclosed in U.S. Pat. No. 4,344,949; ramipril, which may be prepared as disclosed in U.S. Pat. No. 4,587,258; spirapril, which may be prepared as disclosed in U.S. Pat. No. 4,470,972; temocapril, which may be prepared as disclosed in U.S. Pat. No. 4,699,905; and trandolapril, which may be prepared as disclosed in U.S. Pat. No. 4,933,361. The disclosures of all such U.S. Patents are incorporated herein by reference.

Alpha-adrenergic receptor blockers that are within the scope of this invention include, but are not limited to: amosulalol, which may be prepared as disclosed in U.S. Pat. No. 4,217,307; arotinolol, which may be prepared as disclosed in U.S. Pat. No. 3,932,400; dapiprazole, which may be prepared as disclosed in U.S. Pat. No. 4,252,721; doxazosin, which may be prepared as disclosed in U.S. Pat. No. 4,188,390; fenspiride, which may be prepared as disclosed in U.S. Pat. No. 3,399,192; indoramin, which may be prepared as disclosed in U.S. Pat. No. 3,527,761; labetolol, which may be prepared as disclosed above; naftopidil, which may be prepared as disclosed in U.S. Pat. No. 3,997,666; nicergoline, which may be prepared as disclosed in U.S. Pat. No. 3,228,943; prazosin, which may be prepared as disclosed in U.S. Pat. No. 3,511,836; tamsulosin, which may be prepared as disclosed in U.S. Pat. No. 4,703,063; tolazoline, which may be prepared as disclosed in U.S. Pat. No. 2,161,938; trimazosin, which may top prepared as disclosed in U.S. Pat. No. 3,669,968; and yohimbine, which may be isolated from natural sources according to methods well known to those skilled in the art The disclosures of all such U.S. Patents are incorporated herein by reference.

Beta-adrenergic receptor blockers that are within the scope of this invention include, but are not limited to: acebutolol, which may be prepared as disclosed in U.S. Pat. No. 3,857,952; alprenolol, which may be prepared as disclosed in Netherlands Patent Application No. 6,605,692; amosulalol, which may be prepared as disclosed in U.S. Pat. No. 4,217,305; arotinolol, which may be prepared as disclosed in U.S. Pat. No. 3,932,400; atenolol, which may be prepared as disclosed in U.S. Pat. Nos. 3,663,607 or 3,836,671; befunolol, which may be prepared as disclosed in U.S. Pat. No. 3,853,923; betaxolol, which may be prepared as disclosed in U.S. Pat. No. 4,252,984; bevantolol, which may be prepared as disclosed in U.S. Pat. No. 3,857,981; bisoprolol, which may be prepared as disclosed in U.S. Pat. No. 4,171,370; bopindolol, which may prepared as disclosed in U.S. Pat. No. 4,340,541; bucumolol, which may be prepared as disclosed in U.S. Pat. No. 3,663,570; bufetolol, which may be prepared as disclosed in U.S. Pat. No. 3,723,476; bufuralol, which may be prepared as disclosed in U.S. Pat. No. 3,929,836; bunitrolol, which may be prepared as disclosed in U.S. Pat. Nos. 3,940,489 and 3,961,071; buprandolol, which may be prepared as disclosed in U.S. Pat. No. 3,309,406; bubridine hydrochloride, which may be prepared as disclosed in French Patent No. 1,390,056; butofilolol, which may be prepared as disclosed in U.S. Pat. No. 4,252,825; carazolol, which may be prepared as disclosed in German Patent No. 2,240,599; carteolol, which may be prepared as disclosed in U.S. Pat. No. 3,910,924; carvedilol, which may be prepared as disclosed in U.S. Pat. No. 4,503,067; celiprolol, which may be prepared as disclosed in U.S. Pat. No. 4,034,009; cetamolol, which may be prepared as disclosed in U.S. Pat. No. 4,059,622; cloranolol, which may be prepared as disclosed in German Patent No. 2,213,044; dilevalol, which may be prepared as disclosed in Clifton et al., Journal of Medicinal Chemistry, 1982 25, 670; epanolol, which may be prepared as disclosed in European Patent Publication Application No. 41,491; indenolol, which may be prepared as disclosed in U.S. Pat. No. 4,045,482; labetalol, which may be prepared as disclosed in U.S. Pat. No. 4,012,444; levobunolol, which may be prepared as disclosed in U.S. Pat. No. 4,463,176; mepindolol, which may be prepared as disclosed in Seeman et al., Helv. Chim. Acta, 1971, 54 241; metipranolol, which may be prepared as disclosed in Czechoslovakian Patent Application No. 128,471; metoprolol, which may be prepared as disclosed in U.S. Pat. No. 3,873,600; moprolol, which may be prepared as disclosed in U.S. Pat. No. 3,501,769; nadolol, which may be prepared as disclosed in U.S. Pat. No. 3,935, 267; nadoxolol, which may be prepared as disclosed in U.S. Pat. No. 3,819,702; nebivalol, which may be prepared as disclosed in U.S. Pat. No. 4,654,362; nipradilol, which may be prepared as disclosed in U.S. Pat. No. 4,394,382; oxprenolol, which may be prepared as disclosed in British Patent No. 1,077,603; perbutolol, which may be prepared as disclosed in U.S. Pat. No. 3,551,493; pindolol, which may be prepared as disclosed in Swiss Patent Nos. 469,002 and 472,404; practolol, which may be prepared as disclosed in U.S. Pat. No. 3,408,387; pronethalol, which may be prepared as disclosed in British Patent No. 909,357; propranolol, which may be prepared as disclosed in U.S. Pat. Nos. 3,337,628 and 3,520,919; sotalol, which may be prepared as disclosed in Uloth et al., Journal of Medicinal Chemistry, 1966 9, 88; sufinalol, which may be prepared as disclosed in German Patent No. 2,728,641; talindol, which may be prepared as disclosed in U.S. Pat. Nos. 3,935,259 and 4,038,313; tertatolol, which may be. prepared as disclosed in U.S. Pat. No. 3,960,891; tilisolol, which may be prepared as disclosed in U.S. Pat. No. 4,129,565; timolol, which may be prepared as disclosed in U.S. Pat. No. 3,655,663; toliprolol, which may be prepared as disclosed in U.S. Pat. No. 3,432,545; and xibenolol, which may be prepared as disclosed in U.S. Pat. No. 4,018,824. The disclosures of all such U.S. Patents are incorporated herein by reference.

Calcium channel blockers that are within the scope of this invention include, but are not limited to: bepridil, which may be prepared as disclosed in U.S. Pat. No. 3,962, 238 or U.S. Reissue No. 30,577; clentiazem, which may be prepared as disclosed in U.S. Pat. No. 4,567,175; diltiazem, which may be prepared as disclosed in U.S. Pat. No. 3,562, fendiline, which may be prepared as disclosed in U.S. Pat. No. 3,262,977; gallopamil, which may be prepared as disclosed in U.S. Pat. No. 3,261,859; mibefradil, which may be prepared as disclosed in U.S. Pat. No. 4,808,605; prenylamine, which may be prepared as disclosed in U.S. Pat. No. 3,152,173; semotiadil, which may be prepared as disclosed in U.S. Pat. No. 4,786,635; terodiline, which may be prepared as disclosed in U.S. Pat. No. 3,371,014; verapamil, which may be prepared as disclosed in U.S. Pat. No. 3,261,859; aranipine, which may be prepared as disclosed in U.S. Pat. No. 4,572,909; barnidipine, which may be prepared as disclosed in U.S. Pat. No. 4,220,649; benidipine, which may be prepared as disclosed in European Patent Application Publication No. 106,275; cilnidipine, which may be prepared as disclosed in U.S. Pat. No. 4,672,068; efonidipine, which may be prepared as disclosed in U.S. Pat. No. 4,885,284; elgodipine, which may be prepared as disclosed in U.S. Pat. No. 4,952,592; felodipine, which may be prepared as disclosed in U.S. Pat. No. 4,264,611; isradipine, which may be prepared as disclosed in U.S. Pat. No. 4,466,972; lacidipine, which may be prepared as disclosed in U.S. Pat. No. 4,801,599; lercanidipine, which may be prepared as disclosed in U.S. Pat. No. 4,705,797; manidipine, which may be prepared as disclosed in U.S. Pat. No. 4,892,875; nicardipine, which may be prepared as disclosed in U.S. Pat. No. 3,985,758; nifedipine, which may be prepared as disclosed in U.S. Pat. No. 3,485,847; nilvadipine, which may be prepared as disclosed in U.S. Pat. No. 4,338,322; nimodipine, which may be prepared as disclosed in U.S. Pat. No. 3,799,934; nisoldipine, which may be prepared as disclosed in U.S. Pat. No. 4,154,839; nitrendipine, which may be prepared as disclosed in U.S. Pat. No. 3,799,934; cinnarizine, which may be prepared as disclosed in U.S. Pat. No. 2,882,271; flunarizine, which may be prepared as disclosed in U.S. Pat. No. 3,773,939; lidoflazine, which may be prepared as disclosed in U.S. Pat. No. 3,267,104; lomerizine, which may be prepared as disclosed in U.S. Pat. No. 4,663,325; bencyclane, which may be prepared as disclosed in Hungarian Patent No. 151,865; etafenone, which may be prepared as disclosed in German Patent No. 1,265,758; and perhexiline, which may be prepared as disclosed in British Patent No. 1,025,578. The disclosures of all such U.S. Patents are incorporated herein by reference.

The term “vasodilator”, where used herein, is meant to include cerebral vasodilators, coronary vasodilators and peripheral vasodilators. Cerebral vasodilators within the scope of this invention include, but are not limited to: bencyclane, which may be prepared as disclosed above; cinnarizine, which may be prepared as disclosed above; citicoline, which may be isolated from natural sources as disclosed in Kennedy et al., Journal of the American Chemical Society, 1955, 77 250 or synthesized as disclosed in Kennedy, Journal of Biological Chemistry, 1956, 222 185; cyclandelate, which may be prepared as disclosed in U.S. Pat. No. 3,663,597; ciclonicate, which may be prepared as disclosed in German Patent No. 1,910,481; diisopropylamine dichloroacetate, which may be prepared as disclosed in British Patent No. 862,248; ebumamonine, which may be prepared as disclosed in Hermann et al., Journal of the American Chemical Society, 1979, 101, 1540; fasudil, which may be prepared as disclosed in U.S. Pat. No. 4,678,783; fenoxedil, which may be prepared as disclosed in U.S. Pat. No. 3,818,021; flunarizine, which maybe prepared as disclosed in U.S. Pat. No. 3,773,939; ibudilast, which may be prepared as disclosed in U.S. Pat. No. 3,850,941; ifenprodil, which may be prepared as disclosed in U.S. Pat. No. 3,509,164; lomerizine, which may be prepared as disclosed in U.S. Pat. No. 4,663,325; nafronyl, which may be preparedas disclosed in U.S. Pat. No. 3,334,096; nicametate, which may be prepared as disclosed in Blicke et al., Journal of the American Chemical Society, 1942 64 1722; nicergoline, which may be prepared as disclosed above; nimodipine, which may be prepared as disclosed in U.S. Pat. No. 3,799,934; papaverine, which may be prepared as reviewed in Goldberg, Chem. Prod. Chem. News, 1954 17, 371; pentifylline, which may be prepared as disclosed in German Patent No. 860,217; tinofedrine, which may be prepared as disclosed in U.S. Pat. No. 3,563,997; vincamine, which may be prepared as disclosed in U.S. Pat. No. 3,770,724; vinpocetine, which may be prepared as disclosed in U.S. Pat. No. 4,035,750; and viquidil, which may be prepared as disclosed in U.S. Pat. No. 2,500,444. The disclosures of all such U.S. Patents are incorporated herein by reference.

Coronary vasodilators within the scope of this invention include, but are not limited to: amotriphene, which may be prepared as disclosed in U.S. Pat. No. 3,010,965; bendazol, which may be prepared as disclosed in J. Chem. Soc. 1958, 2426; benfurodil hemisuccinate, which may be prepared as disclosed in U.S. Pat. No. 3,355,463; benziodarone, which may be prepared as disclosed in U.S. Pat. No. 3,012,042; chloracizine, which may be prepared as disclosed in British Patent No. 740,932; chromonar, which may be prepared as disclosed in U.S. Pat. No. 3,282,938; clobenfural, which may be prepared as disclosed in British Patent No. 1,160,925; clonitrate, which may be prepared from propanediol according to methodswell known to those skilled in the art, e.g., see Annalen, 1870, 155, 165; cloricromen, which may be prepared as disclosed in U.S. Pat. No. 4,452,811; dilazep, which may be prepared as disclosed in U.S. Pat. No. 3,532,685; dipyridamole, which maybe prepared as disclosed in British Patent No. 807,826; droprenilamine, which maybe prepared as disclosed in German Patent No. 2,521,113; efloxate, which may be prepared as disclosed in British Patent Nos. 803,372 and 824,547; erythrityltetranitrate, which may be prepared by nitration of erythritol according to methods well-known to those skilled in the art; etafenone, which may be prepared as disclosed in German Patent No. 1,265,758; fendiline, which may be prepared as disclosed in U.S. Pat. No. 3,262,977; floredil, which may be prepared as disclosed in German Patent No. 2,020,464; ganglefene, which may be prepared as disclosed in U.S.S.R. Patent No. 115,905; hexestrol, which may be prepared as disclosed in U.S. Pat. No. 2,357,985; hexobendine, which may be prepared as disclosed in U.S. Pat. No. 3,267,103; itramin tosylate, which may be prepared as disclosed in Swedish Patent No. 168,308; khellin, which may be prepared as disclosed in Baxter et al., Journal of the Chemical Society, 1949, S 30; lidoflaznve, which may be prepared as disclosed in U.S. Pat. No. 3,267,104; mannitol hexanitrate, which may be prepared by the nitration of mannitol according to methods well-known to those skilled in the art; medibazine, which may be prepared as disclosed in U.S. Pat. No. 3,119,826; nitroglycerin; pentaerythritol tetranitrate, which may be prepared by the nitration of pentaerythritol according to methods well-known to those skilled in the art; pentrinitrol, which may be prepared as disclosed in German Patent No. 638,422-3; perhexilline, which may be prepared as disclosed above; pimefylline, which may be prepared as disclosed in U.S. Pat. No. 3,350,400; prenylamine, which may be prepared as disclosed in U.S. Pat. No. 3,152,173; propatyl nitrate, which may be prepared as disclosed in French Patent No. 1,103,113; trapidil, which may be prepared as disclosed in East German Patent No. 55,956; tricromyl, which may be prepared as disclosed in U.S. Pat. No. 2,769,015; trimetazidine, which may be prepared as disclosed in U.S. Pat. No. 3,262,852; trolnitrate phosphate, which maybe prepared by nitration of triethanolamine followed by precipitation with phosphoric acid according to methods well-known to those skilled in the art; visnadine, which may be prepared as disclosed in U.S. Pat. Nos. 2,816,118 and 2,980,699. The disclosures of all such U.S. Patents are incorporated herein by reference.

Peripheral vasodilators within the scope of this invention include, but are not limited to: aluminum nicotinate, which may be prepared as disclosed in U.S. Pat. No. 2,970,082; bamethan, which may be prepared as disclosed in Corrigan et al., Journal of the American Chemical Society, 1945, 67 1894; bencyclane, which may be prepared as disclosed above; betahistine, which may be prepared as disclosed in Walter et al.; Journal of the American Chemical Society, 1941, 63, 2771; bradykinin, which may be prepared as disclosed in Hamburg et al., Arch. Biochem. Biophys., 1958, 76 252; brovincamine, which may be prepared as disclosed in U.S. Pat. No. 4,146,643; bufeniode, which may be prepared as disclosed in U.S. Pat. No. 3,542,870; buflomedil, which may be prepared as disclosed in U.S. Pat. No. 3,895,030; butalamine, which may be prepared as disclosed in U.S. Pat. No. 3,338,899; cetiedil, which may be prepared as disclosed in French Patent Nos. 1,460,571; ciclonicate, which may be prepared as disclosed in German Patent No. 1910,481; cinepazide, which may be prepared as disclosed in Belgian Patent No. 730,345; cinnarizine, which may be prepared as disclosed above; cyclandelate, which may be prepared as disclosed above; diisopropylamine dichloroacetate, which maybe prepared as disclosed above; eledoisin, which may be prepared as disclosed in British Patent No. 984,810; fenoxedil, which may be prepared as disclosed above; flunarizine, which may be prepared as disclosed above; hepronicate, which may be prepared as disclosed in U.S. Pat. No. 3,384,642; ifenprodil, which may be prepared as disclosed above; iloprost, which may be prepared as disclosed in U.S. Pat. No. 4,692,464; inositol niacinate, which may be prepared as disclosed in Badgett et al., Journal of the American Chemical Society, 1947 69, 2907; isoxsuprine, which may be prepared as disclosed in U.S. Pat. No. 3,056,836; kallidin, which may be prepared as disclosed in Biochem. Biophys. Res. Commun.,1961, 6, 210; kallikrein, which may be prepared as disclosed in German Patent No. 1,102,973; moxisylyte, which may be prepared as disclosed in German Patent No. 905,738; nafronyl, which may be prepared as disclosed above; nicametate, which may be prepared as disclosed above; nicergoline, which may be prepared as disclosed above; nicofuranose, which may be prepared as disclosed in Swiss Patent No. 366,523; nylidrin, which may be prepared as disclosed in U.S. Pat. Nos. 2,661,372 and 2,661,373; pentifylline, which may be prepared as disclosed above; pentoxifylline, which may be prepared as disclosed in U.S. Pat. No. 3,422,107; piribedil, which may be prepared as disclosed in U.S. Pat. No. 3,299,067; prostaglandin El, which may be prepared by any of the methods referenced in the Merck Index, Twelfth Edition, Budaveri, Ed., New Jersey, 1996, p. 1353; suloctidil, which may be prepared as disclosed in German Patent No. 2,334,404; tolazoline, which may be prepared as disclosed in U.S. Pat. No. 2,161,938; and xanthinolniacinate, which may be prepared as disclosed in German Patent No. 1,102,750 or Korbonits et al., Acta. Pharm. Hung., 1968, 38, 98. The disclosures of all such U.S. Patents are incorporated herein by reference.

The term “diuretic”, within the scope of this invention, is includes, but is not limited to, diuretic benzothiadiazine derivatives, diuretic organomercurials, diuretic purines, diuretic steroids (including diuretic steroids having no substantial activity as an aldosterone receptor antagonist), diuretic sulfonamide derivatives, diuretic uracils and other diuretics such as amanozine, which may be prepared as disclosed in Austrian Patent No. 168,063; amiloride, which may be prepared as disclosed in Belgian Patent No. 639,386; arbutin, which may be prepared as disclosed in Tschitschibabin, Annalen,1930, 478, 303; chlorazanil, which may be prepared as disclosed in Austrian Patent No. 168,063; ethacrynic acid, which may be prepared as disclosed in U.S. Pat. No. 3,255,241; etozolin, which may be prepared as disclosed in U.S. Pat. No. 3,072,653; hydracarbazine, which may be prepared as disclosed in British Patent No. 856,409; isosorbide, which may be prepared as disclosed in U.S. Pat. No. 3,160,641; mannitol; metochalcone, which may be prepared as disclosed in Freudenberg et al., Ber., 1957, 90, 957; muzolimine, which may be prepared as disclosed in U.S. Pat. No. 4,018,890; perhexiline, which may be prepared as disclosed above; ticrynafen, which may be prepared as disclosed in U.S. Pat. No. 3,758,506; triamterene which may be prepared as disclosed in U.S. Pat. No. 3,081,230; and urea. The disclosures of all such U.S. Patents are incorporated herein by reference.

Diuretic benzothiadiazine derivatives within the scope of this invention include, but are not limited to: althiazide, which may be prepared as disclosed in British Patent No. 902,658; bendroflumethiazide, which may be prepared as disclosed in U.S. Pat. No. 3,265,573; benzthiazide, McManus et al., 136th Am. Soc. Meeting (Atlantic City, September 1959), Abstract of papers, pp 13-0; benzylhydrochlorothiazide, which may be prepared as disclosed in U.S. Pat. No. 3,108,097; buthiazide, which may be prepared as disclosed in British Patent Nos. 861,367 and 885,078; chlorothiazide, which may be prepared as disclosed in U.S. Pat. Nos. 2,809,194 and 2,937,169; chlorthalidone, which may be prepared as disclosed in U.S. Pat. No. 3,055,904; cyclopenthiazide, which may be prepared as disclosed in Belgian Patent No. 587,225; cyclothiaide, which may be prepared as disclosed in Whitehead et al., Journal of Organic Chemistry, 1961, 26, 2814; epithiazide, which may be prepared as disclosed in U.S. Pat. No. 3,009,911; ethiazide, which may be prepared as disclosed in British Patent No. 861,367; fenquizone, which may be prepared as disclosed in U.S. Pat. No. 3,870,720; indapamide, which may be prepared as disclosed in U.S. Pat. No. 3,565,911; hydrochlorothiazide, which may be prepared as disclosed in U.S. Pat. No. 3,164,588; hydroflumethiazide, which may be prepared as disclosed in U.S. Pat. No. 3,254,076; methyclothiazide, which may be prepared as disclosed in Close et al., Journal of the American Chemical Society, 1960, 82, 1132; meticrane, which may be prepared as disclosed in French Patent Nos. M2790 and 1,365,504; metolazone, which may be prepared as disclosed in U.S. Pat. No. 3,360,518; paraflutizide, which may be prepared as disclosed in Belgian Patent No. 620,829; polythiazide, which may be prepared as disclosed in U.S. Pat. No. 3,009,911; quinethazone, which may be prepared as disclosed in U.S. Pat. No. 2,976,289; teclothiazide, which may be prepared as disclosed in Close et al., Journal of the American Chemical Society, 1960, 82, 1132; and trichlormethiazide, which may be prepared as disclosed in deStevens et al., Experientia, 1960, 16, 113. The disclosures of all such U.S. Patents are incorporated herein by reference.

Diuretic sulfonamide derivatives within the scope of this invention include, but are not limited to: acetazolamide, which may be prepared as disclosed in U.S. Pat. No. 2,980,679; ambuside, which may be prepared as disclosed in U.S. Pat. No. 3,188,329; azosernide, which may be prepared as disclosed in U.S. Pat. No. 3,665,002; bumetanide, which may be prepared as disclosed in U.S. Pat. No. 3,634,583; butazolamide, which may be prepared as disclosed in British Patent No. 769,757; chloraminophenamide, which may be prepared as disclosed in U.S. Pat. Nos. 2,809,194, 2,965,655 and 2,965,656; clofenamide, which may be prepared disclosed in Olivier, Rec. Trav. Chim., 1918, 37 307; clopamide, which may be prepared as disclosed in U.S. Pat. No. 3,459,756; clorexolone, which may be prepared as disclosed in U.S. Pat. No. 3,183,243; disulfamide, which may be prepared as disclosed in British Patent No. 851,287; ethoxolamide, which may be prepared as disclosed in British Patent No. 795,174; furosemide, which may be prepared as disclosed in U.S. Pat. No. 3,058,882; mefruside, which may be prepared as disclosed in U.S. Pat. No. 3,356,692; methazolamide, which may be prepared as disclosed in U.S. Pat. No. 2,783,241; piretanide, which may be prepared as disclosed in U.S. Pat. No. 4,010,273; torasemide, which may be prepared as disclosed in U.S. Pat. No. 4,018,929; tripamide, which may be prepared as disclosed in Japanese Patent No. 73 05,585; and xipamide, which maybe prepared, as disclosed in U.S. Pat. No. 3,567,777. The disclosures of all such U.S. Patents are incorporated herein by reference.

In one embodiment the aldosterone receptor antagonist and endothelin receptor antagonist and/or ECE inhibitor can be administered in combination with a renin inhibitor.

In another embodiment the aldosterone receptor antagonist and endothelin receptor antagonist and/or ECE inhibitor can be administered in combination with an angiotensin I antagonist.

In another embodiment the aldosterone receptor antagonist and endothelin receptor antagonist and/or ECE inhibitor can be administered in combination with an angiotensin II antagonist.

In another embodiment the aldosterone receptor antagonist and endothelin receptor antagonist and/or ECE inhibitor can be administered in combination with an angiotensin converting enzyme inhibitor.

In another embodiment the aldosterone receptor antagonist and endothelin receptor antagonist and/or ECE inhibitor can be administered in combination with an alpha-adrenergic receptor blocker.

In another embodiment the aldosterone receptor antagonist and endothelin receptor antagonist and/or ECE inhibitor can be administered in combination with a beta-adrenergic receptor blocker.

In another embodiment the aldosterone receptor antagonist and endothelin receptor antagonist and/or ECE inhibitor can be administered in combination with a calcium channel blocker.

In another embodiment the aldosterone receptor antagonist and endothelin receptor antagonist and/or ECE inhibitor can be administered in combination with a neutral endopeptidase inhibitors (such as omapatrilat).

In another embodiment the aldosterone receptor antagonist and endothelin receptor antagonist and/or ECE inhibitor can be administered in combination with an aldosterone receptor antagonist (such as eplerenone and spironolactone).

In another embodiment the aldosterone receptor antagonist and endothelin receptor antagonist and/or ECE inhibitor can be administered in combination with a vasodilator.

In another embodiment the aldosterone receptor antagonist and endothelin receptor antagonist and/or ECE inhibitor can be administered in combination with a diuretic.

In another embodiment the aldosterone receptor antagonist and endothelin receptor antagonist and/or ECE inhibitor can be administered in combination with a member of the group consisting of lipid-lowering drugs (including apical sodium bile acid transport inhibitors, cholesterol absorption inhibitors, fibrates, niacin, statins, cholesteryl ester transfer protein inhibitors, and bile acid sequestrants).

In another embodiment the aldosterone receptor antagonist and endothelin receptor antagonist and/or ECE inhibitor can be administered in combination with anti-oxidants (including vitamin E and probucol).

In another embodiment the aldosterone receptor antagonist and endothelin receptor antagonist and/or ECE inhibitor can be administered in combination with a IIb/IIIa antagonist.

Administration of a aldosterone receptor antagonist and endothelin receptor antagonist and/or ECE inhibitor also can be effected in combination with one or more of non-drug therapies, such as non-drug therapies associated with the treatment of restenosis. For example, conventional treatment of restenosis resulting from angioplasty includes therapies such as exposing the artery at the site of injury to a source of radiation to inhibit restrictive neointima growth and inserting an endolumenal stent at the site of angioplasty.

In one embodiment, the aldosterone receptor antagonist and endothelin receptor antagonist and/or ECE inhibitor can be administered in combination with exposure of an angioplastied artery at the site of injury to a source of radiation to inhibit restrictive neointima growth. Although radiation monotherapy has been used to prevent restenosis after angioplasty, Powers et al., Int. J. Radiat. Oncol. Biol, Vol. 45(3), pp. 753-759 (Oct. 1, 1999), report findings in a study involving a canine model that indicate that adventitial fibrosis increases with increasing dose of radiation and can contribute to adverse late vascular remodeling. The proposed combination therapy would permit the use of dosages of radiation below conventional monotherapeutic dosages of radiation and would result in fewer side-effects or adverse effects relative to such radiation monotherapy.

In another embodiment, the stent itself comprises the aldosterone receptor antagonist and endothelin receptor antagonist and/or ECE inhibitor and is used as a carrier to effect local delivery of the aldosterone receptor antagonist and endothelin receptor antagonist and/or ECE inhibitor to the injured vessel. The aldosterone receptor antagonist and endothelin receptor antagonist and/or ECE inhibitor is coated on, adsorbed on, affixed to or present on the surface of the stent or is otherwise present in or on the matrix of the stent, either alone or in combination with other active drugs and pharmaceutically acceptable carriers, adjuvants, binding agents and the like. The stent preferably comprises the aldosterone receptor antagonist and endothelin receptor antagonist and/or ECE inhibitor in the form of an extended release composition that provides for release of the antagonists over an extended period of time.

Aldosterone Receptor Antagonist/Endothelin Receptor Antagonist Kits

The present invention further comprises kits comprising one or more aldosterone receptor antagonists and one or more endothelin receptor antagonists that are suitable for use in performing the methods of treatment and/or prevention described above. In one embodiment, the kit contains a first dosage form comprising one or more of the aldosterone receptor antagonists identified in Table 1 and a second dosage form comprising one or more of the endothelin receptor antagonists identified in Tables 2, 3, or 4 in quantities sufficient to carry out the methods of the present invention. The first dosage form and the second dosage form together comprise a therapeutically effective amount of the inhibitors for the treatment or prevent of a pathological condition.

In another embodiment, the kit contains a first dosage form comprising the aldosterone receptor antagonist spironolactone and a second dosage form comprising an endothelin receptor antagonist identified in Table 2, 3 or 4 in quantities sufficient to carry out the methods of the present invention.

In another embodiment, the kit contains a first dosage form comprising the aldosterone receptor antagonist eplerenone and a second dosage form comprising an endothelin receptor antagonist identified in Table 2, 3 or 4 in quantities sufficient to carry out the methods of the present invention.

In a further embodiment, the kit contains a first dosage form comprising the aldosterone receptor antagonist eplerenone, a second dosage form comprising an endothelin receptor antagonist identified in Table 2, 3, or 4, and a third dosage of an active drug in quantities sufficient to carry out the methods of the present invention. Examples of active drugs which may be contained in the kit include, but are not limited to active drugs selected from the group consisting of renin inhibitors, angiotensin I antagonists, angiotensin II antagonists, angiotensin converting enzyme inhibitors, alpha-adrenergic receptor blockers, beta-adrenergic receptor blockers, calcium channel blockers, neutral endopeptidase inhibitors, vasodilators, diuretics, cyclooxygenase-1 inhibitors, apical sodium bile acid transport inhibitors, cholesterol absorption inhibitors, fibrates, niacin, statins, cholesteryl ester transfer protein inhibitors, bile acid sequestrants, anti-oxidants, vitamin E, probucol, and IIb/IIIa antagonists.

In still another embodiment, the kit contains a first dosage form comprising one or more of the aldosterone receptor antagonists identified in Table 1, a second dosage form comprising an endothelin receptor antagonist identified in Table 2, 3, or 4, and a third dosage form comprising an ECE inhibitor identified in Table 5 in quantities sufficient to carry out the methods of the present invention. The first dosage form, second dosage form, and third dosage form together comprise a therapeutically effective amount of the antagonists and inhibitors for the prophylaxis and/or treatment of pathological condition such as hypertension, cardiovascular disease, renal dysfunction, edema, cerebrovascular disease, insulinopathies, and the like.

In another embodiment, the kit contains a first dosage form comprising the aldosterone receptor antagonist spironolactone, a second dosage form comprising an endothelin receptor antagonist identified in Table 2, 3, or 4, and a third dosage form comprising an ECE inhibitor identified in Table 5 in quantities sufficient to carry out the methods of the present invention.

In another embodiment, the kit contains a first dosage form comprising the aldosterone receptor antagonist eplerenone, a second dosage form comprising an endothelin receptor antagonist identified in Table 2, 3, or 4, and a third dosage form comprising an ECE inhibitor identified in Table 5 in quantities sufficient to carry out the methods of the present invention. In a further embodiment, the kit contains a first dosage form comprising the aldosterone receptor antagonist eplerenone a second dosage form comprising an endothelin receptor antagonist identified in Table 2, 3, or 4, and a third dosage form comprising an ECE inhibitor identified in Table 5.

In a further embodiment, the kit contains a first dosage form comprising the aldosterone receptor antagonist eplerenone, a second dosage form comprising an endothelin receptor antagonist identified in Table 2, 3, or 4, and a third dosage form comprising an ECE inhibitor identified in Table 5, and a fourth dosage of an active drug in quantities sufficient to carry out the methods of the present invention. Examples of active drugs which may be contained in the kit include, but are not limited to active drugs selected from the group consisting of renin inhibitors, angiotensin I antagonists, angiotensin II antagonists, angiotensin converting enzyme inhibitors, alpha-adrenergic receptor blockers, beta-adrenergic receptor blockers, calcium channel blockers, neutral endopeptidase inhibitors, vasodilators, diuretics, cyclooxygenase-1 inhibitors, apical sodium bile acid transport inhibitors, cholesterol absorption inhibitors, fibrates, niacin, statins, cholesteryl ester transfer protein inhibitors, bile acid sequestrants, anti-oxidants, vitamin E, probucol, and IIb/IIIa antagonists.

Aldosterone Receptor Antagonist/ECE Inhibitor Kits

The present invention further comprises kits comprising one or more aldosterone receptor antagonists and one or more ECE inhibitors that are suitable for use in performing the methods of treatment and/or prevention described above. In one embodiment, the kit contains a first dosage form comprising one or more of the aldosterone receptor antagonists identified in Table 1 and a second dosage form comprising one or more of the ECE inhibitors identified in Table 5 in quantities sufficient to carry out the methods of the present invention. The first dosage form and the second dosage form together comprise a therapeutically effective amount of the inhibitors for the treatment or prevent of a pathological condition.

In another embodiment, the kit contains a first dosage form comprising the aldosterone receptor antagonist spironolactone and a second dosage form comprising an ECE inhibitor identified in Table 5 in quantities sufficient to carry out the methods of the present invention.

In another embodiment, the kit contains a first dosage form comprising the aldosterone receptor antagonist eplerenone and a second dosage form comprising an ECE inhibitor identified in Table 5 in quantities sufficient to carry out the methods of the present invention.

In a further embodiment, the kit contains a first dosage form comprising the aldosterone receptor antagonist eplerenone, a second dosage form comprising an ECE inhibitor identified in Table 5, and a third dosage of an active drug in quantities sufficient to carry out the methods of the present invention. Examples of active drugs which may be contained in the kit include, but are not limited to active drugs selected from the group consisting of renin inhibitors, angiotensin I antagonists, angiotensin II antagonists, angiotensin converting enzyme inhibitors, alpha-adrenergic receptor blockers, beta-adrenergic receptor blockers, calcium channel blockers, neutral endopeptidase inhibitors, vasodilators, diuretics, cyclooxygenase-1 inhibitors, apical sodium bile acid transport inhibitors, cholesterol absorption inhibitors, fibrates, niacin, statins, cholesteryl ester transfer protein inhibitors, bile acid sequestrants, anti-oxidants, vitamin E, probucol, and IIb/IIIa antagonists.

Dosage and Treatment Regimen

Aldosterone Receptor Antagonist Dosing

The amount of aldosterone receptor antagonist that is administered and the dosage regimen for the methods of this invention depend on a variety of factors, including the age, weight, sex and medical condition of the subject, the severity of the pathological conditions, the route and frequency of administration, and the particular aldosterone receptor antagonist employed, and thus may vary widely. A daily dose administered to a subject of about 0.001 to 30 mg/kg body weight, or between about 0.005 and about 20 mg/kg body weight, or between about 0.01 and about 15 mg/kg body weight, or between about 0.05 and about 10 mg/kg body weight, or between about 0.1 to 5 mg/kg body weight, may be appropriate. The amount of aldosterone receptor antagonist that is administered to a human subject typically will range from about 0.1 to 2000 mg, or from about 0.5 to 500 mg, or from about 0.75 to 250 mg, or from about 1 to 100 mg. A daily dose of aldosterone receptor antagonist that produces no substantial diuretic and/or anti-hypertensive effect in a subject is specifically embraced by the present method. The daily dose can be administered in one to four doses per day.

Dosing of the aldosterone receptor antagonist can be determined and adjusted based on measurement of blood pressure or appropriate surrogate markers (such as natriuretic peptides and other surrogate markers discussed below). Blood pressure and/or surrogate marker levels after administration of the aldosterone receptor antagonist can be compared against the corresponding baseline levels prior to administration of the aldosterone receptor antagonist to determine efficacy of the present method and titrated as needed. Non-limiting examples of surrogate markers useful in the method are surrogate markers for renal and cardiovascular disease.

Prophylactic Dosing

It is beneficial to administer the aldosterone receptor antagonist prophylatically, prior to a diagnosis of inflammation-related cardiovascular disorders, and to continue administration of the aldosterone receptor antagonist during the period of time the subject is susceptible to the inflammation-related cardiovascular disorders. Individuals with no remarkable clinical presentation but that are nonetheless susceptible to pathological conditions therefore can be placed upon a prophylactic dose of an aldosterone receptor antagonist compound. Such prophylactic doses of the aldosterone receptor antagonist may, but need not, be lower than the doses used to treat the specific condition or disorder of interest.

Cardiovascular Pathology Dosing

Dosing to treat pathologies of cardiovascular function can be determined and adjusted based on measurement of blood concentrations of natriuretic peptides. Natriuretic peptides are a group of structurally similar but genetically distinct peptides that have diverse actions in cardiovascular, renal, and endocrine homeostasis. Atrial natriuretic peptide (“ANP”) and brain natriuretic peptide (“BNP”) are of myocardial cell origin and C-type natriuretic peptide (“CNP”) is of endothelial origin. ANP and BNP bind to the natriuretic peptide-A receptor (“NPR-A”), which, via 3′,5′-cyclic guanosine monophosphate (cGMP), mediates natriuresis, vasodilation, renin inhibition, antimitogenesis, and lusitropic properties. Elevated natriuretic peptide levels in the blood, particularly blood BNP levels, generally are observed in subjects under conditions of blood volume expansion and after vascular injury such as acute myocardial infarction and remain elevated for an extended period of time after the infarction. (Uusimaa et al.: Int. J. Cardiol 1999; 69: 5-14).

A decrease in natriuretic peptide level relative to the baseline level measured prior to administration of the aldosterone receptor antagonist indicates a decrease in the pathologic effect of aldosterone and therefore provides a correlation with inhibition of the pathologic effect. Blood levels of the desired natriuretic peptide level therefore can be compared against the corresponding baseline level prior to administration of the aldosterone receptor antagonist to determine efficacy of the present method in treating pathological conditions. Based upon such natriuretic peptide level measurements, dosing of the aldosterone receptor antagonist can be adjusted to reduce the cardiovascular adverse condition or disorder.

Similarly, cardiac pathological conditions can also be identified, and the appropriate dosing determined, based on circulating and urinary cGMP Levels. An increased plasma level of cGMP parallels a fall in mean arterial pressure. Increased urinary excretion of cGMP is correlated with the natriuresis.

Cardiac pathological conditions also can be identified by a reduced ejection fraction or the presence of myocardial infarction or heart failure or left ventricular hypertrophy. Left ventricular hypertrophy can be identified by echo-cardiogram or magnetic resonance imaging and used to monitor the progress of the treatment and appropriateness of the dosing.

In another embodiment of the invention, therefore, the methods of the present invention can be used to reduce natriuretic peptide levels, particularly BNP levels, thereby also treating related cardiovascular pathological conditions.

Renal Pathology Dosing

Dosing to treat pathological conditions of renal function can be determined and adjusted based on measurement of proteinuria, microalbuminuria, decreased glomerular filtration rate (GFR), or decreased creatinine clearance. Proteinuria is identified by the presence of greater than 0.3 g of urinary protein in a 24 hour urine collection. Microalbuminuria is identified by an increase in immunoassayable urinary albumin. Based upon such measurements, dosing of the aldosterone receptor antagonist can be adjusted to reduce the renal adverse condition or disorder.

Neuropathy Pathology Dosing

Neuropathy, especially peripheral neuropathy, can be identified by and dosing adjustments based on, neurologic exam of sensory deficit or sensory motor ability.

Retinopathy Pathology Dosing

Retinopathy can be identified by, and dosing adjustments based on, opthamologic exam.

Endothelin Receptor Antagonist Dosing

The amount of endothelin receptor antagonist that is administered and the dosage regimen for the methods of this invention also depend on a variety of factors, including the age, weight, sex and medical condition of the subject, the severity of the pathological condition, the route and frequency of administration, and the particular endothelin receptor antagonist employed, and thus may vary widely. A daily dose administered to a subject of about 0.001 to 100 mg/kg body weight, or between about 0.005 and about 60 mg/kg body weight, or between about 0.01 and about 50 mg/kg body weight, or between about 0.05 and about 30 mg/kg body weight, or between about 0.1 to 20 mg/kg body weight, may be appropriate.

The amount of endothelin receptor antagonist that is administered to a human subject typically will range from about 0.1 to 2400 mg, or from about 0.5 to 2000 mg, or from about 0.75 to 1000 mg, or from about 1.0 to 600 mg, or from about 5.0 to 300 mg, or from about 10.0 to 100 mg. A daily dose of endothelin receptor antagonist that produces no substantial diuretic and/or anti-hypertensive effect in a subject is specifically embraced by the present method. The daily dose can be administered in one to six doses per day.

Combination Therapy Dosages

It is understood, however, that the specific dose level for each patient will depend upon a variety of factors including the activity of the specific inhibitors employed, the age, body weight, general health, sex, diet, time of administration, rate of excretion, inhibitor combination selected, the severity of the particular pathological condition being treated, and the form of administration. Appropriate dosages can be determined in trials. The ratio of aldosterone receptor antagonist to endothelin receptor antagonist and/or ECE inhibitor (weight/weight), however, typically will range from about 1:100 to about 100:1, or about 1:50 to about 50:1, or about 1:20 to about 20:1, or about 1:5 to about 5:1, or about 1:2 to about 2:1.

The total daily dose of each drug can be administered to the patient in a single dose, or in proportionate multiple subdoses. Subdoses can be administered two to six times per day. Doses can be in immediate release form or sustained release form effective to obtain desired results. Single dosage forms comprising the aldosterone receptor antagonist and the endothelin receptor antagonist and/or ECE inhibitor may be used where desirable.

Dosage Regimen

As noted above, the dosage regimen to prevent, treat, give relief from, or ameliorate a pathological condition, with the combinations and compositions of the present invention is selected in accordance with a variety of factors. These factors include the type, age, weight, sex, diet, and medical condition of the patient, the type and severity of the disease, the route of administration, pharmacological considerations such as the activity, efficacy, pharmacokinetics and toxicology profiles of the particular inhibitors employed, whether a drug delivery system is utilized, and whether the inhibitors are administered with other ingredients. Thus, the dosage regimen actually employed may vary widely and therefore deviate from the preferred dosage regimen set forth above.

Initial treatment of a patient suffering from a pathological condition such as hypertension, cardiovascular disease, renal dysfunction, edema, cerebrovascular disease, insulinopathies, and the like can begin with the dosages indicated above. Treatment generally should be continued as necessary over a period of several weeks to several months or years until the pathological condition has been controlled or eliminated. Patients undergoing treatment with the combinations or compositions disclosed herein can be routinely monitored to determine treatment effectiveness. For example, in treating specific hypertension or cardiovascular pathological conditions, measuring blood pressure, or other indicator of the pathological condition by any of the methods well-known in the art, may be conducted to determine the effectiveness of the combination therapy. Continuous analysis of such data permits modification of the treatment regimen during therapy so that optimal effective amounts of each type of inhibitor are administered at any time, and so that the duration of treatment can be determined as well. In this way, the treatment regimen/dosing schedule can be rationally modified over the course of therapy so that the lowest amount of aldosterone receptor antagonist and endothelin receptor antagonist and/or ECE inhibitor that together exhibit satisfactory effectiveness is administered, and so that administration is continued only so long as is necessary to successfully treat the pathological condition.

In combination therapy, administration of the aldosterone receptor antagonist and the endothelin receptor antagonist and/or ECE inhibitor may take place in sequence as part of a timed relationship in separate formulations, or may be accomplished by simultaneous administration in a single formulation or separate formulations.

When administered in a seqencence, the timed relationship between administration of the aldosterone receptor antagonist and NEP inhibitor (and optionally ACE inhibitor) is less than 24 hours. In another embodiment the timed relationship is less than 12 hours. In another embodiment the timed relationship is less than 8 hours. In another embodiment the timed relationship is less than 6 hours. In another embodiment the timed relationship is less than 4 hours. In another embodiment the timed relationship is less than 1 hour. In another embodiment the timed relationship is less than thirty minutes. In another embodiment the timed relationship is less than ten minutes. In another embodiment the timed relationship is less than one minute.

Administration may be accomplished by any appropriate route, with oral administration being preferred. The dosage units used may with advantage contain one or more aldosterone receptor antagonist and one or more endothelin receptor antagonist and/or ECE inhibitors in the amounts described above.

Dosing for oral administration may be with a regimen calling for a single daily dose, for multiple, spaced doses throughout the day, for a single dose every other day, for a single dose every several days, or other appropriate regimens. The aldosterone receptor antagonist and the endothelin receptor antagonist and/or ECE inhibitor used in the combination therapy may be administered simultaneously, either in a combined dosage form or in separate dosage forms intended for substantially simultaneous oral administration. The aldosterone receptor antagonists and the endothelin receptor antagonist and/or ECE inhibitors also may be administered sequentially, with either inhibitor being administered by a regimen calling for two-step ingestion. Thus, a regimen may call for sequential administration of the aldosterone receptor antagonist and the endothelin receptor antagonist and/or ECE inhibitor with spaced-apart ingestion of these separate, active agents. The time period between the multiple ingestion steps may range from a few minutes to several hours, depending upon the properties of each active agent such as potency, solubility, bioavailability, plasma half-life and kinetic profile of the inhibitor, as well as depending upon the age and condition of the patient. Dose timing may also depend on the circadian or other rhythms for the pathological effects of agents, such as aldosterone, which may be optimally blocked at the time of their peak concentration. The combination therapy, whether administration is simultaneous, substantially simultaneous, or sequential, may involve a regimen calling for administration of the aldosterone receptor antagonist by oral route and the endothelin receptor antagonist and/or ECE inhibitor by intravenous route. Whether these active agents are administered by oral or intravenous route, separately or together, each such active agent will be contained in a suitable pharmaceutical formulation of pharmaceutically acceptable excipients, diluents or other formulations components. Examples of suitable pharmaceutically acceptable formulations are given above.

The aldosterone receptor antagonist and the endothelin receptor antagonist and/or ECE inhibitor may also be administered by injection as a composition wherein, for example, saline, dextrose or water may be used as a suitable carrier. A suitable daily dose of each active component is from about 0.01 to 30 mg/kg body weight injected per day in multiple doses depending on the disease being treated. A preferred daily dose is from about 1 to 15 mg/kg body weight. Compounds indicated for prophylactic therapy will preferably be administered in a daily dose generally in a range from about 0.01 mg to about 30 mg per kilogram of body weight per day. A more preferred dosage will be a range from about 1 mg to about 15 mg per kilogram of body weight. Most preferred is a dosage in a range from about 1 to about 10 mg per kilogram of body weight per day. A suitable dose can be administered, in multiple sub-doses per day.

Biological Evaluation

Human congestive heart failure (CHF) is a complex condition usually initiated by vascular hypertension or a myocardial infarction (MI). In order to determine the probable effectiveness of a combination therapy for CHF, it is important to determine the potency of components in several assays. Accordingly, in Assays “A” and “B”, the endothelin receptor antagonists or ECE inhibitor activity can be determined. In Assays “C” and “D” a method is described for evaluating a combination therapy of the invention, namely, an endothelin receptor antagonist or ECE inhibitor and an epoxy-steroidal aldosterone receptor antagonist. The efficacy of the individual drugs, eplerenone, and an endothelin receptor antagonist or ECE inhibitor, and of these drugs given together at various doses, are evaluated in rodent models of hypertension and CHF using surgical alterations to induce either hypertension or an MI. The methods of such assays are described below.

In addition, clinical trials can be used to evaluate aldosterone receptor antagonist therapy in humans. Numerous examples of such therapeutic tests have been published, including those of the RALES 003 study described in American Journal of Cardiology 78, 902-907 (1996) or the RALES 004 study described in New England Journal of Medicine 341, 709-717 (1999).

Assay A: In Vitro Vascular Smooth Muscle-Response

Thoracic aortas, removed from male Sprague-Dawley rats (350-550 g), are dissected free from surrounding connective tissue, and cut into ring segments each about 2-3 mm long. Smooth muscle rings are mounted for isometric tension recording in an organ bath filled with 10 mL of Krebs-Henseleit (K—H) solution, pH 7.4. This bathing solution is maintained at 37° C. and bubbled with 95% O₂/5% CO₂. The strips are stretched to a tension of 2 g and allowed to equilibrate. Isometric tension changes are monitored using an isometric transducer and recorded on a potentiometric recorder. A precontraction is produced by adding a catecholamine or by changing the solution to 30 mM K⁺. Contraction is maintained for 30 minutes, and the preparation washed with Krebs-Henseleit solution. After sixty minutes, contraction is induced in the same manner as described above. Subsequently, a test compound is added to obtain a concentration-response curve. Taking the precontraction value as 100%, the concentration of the drug at which the contraction is relaxed to 50% is the IC₅₀.

Assay B: In Vivo Intragastric Pressor Assay Response

Male Sprague-Dawley rats weighing 225-300 grams are anesthetized with methohexital (30 mg/kg, i.p.) and catheters were implanted into the femoral artery and vein. The catheters are tunneled subcutaneously to exit dorsally, posterior to the head and between the scapulae. The catheters are filled with heparin (1000 units/ml of saline). The rats are returned to their cage and allowed regular rat chow and water ad libitum. After full recovery from surgery (3-4 days), rats are placed in Lucite holders and the arterial line is connected to a pressure transducer. Arterial pressure is recorded on a Gould polygraph (mmHg). Epinephrine or norepinephrine is administered as a 30 ng/kg bolus via the venous catheter delivered in a 50 μl volume with a 0.2 ml saline flush. The pressor response in mm Hg is measured by the difference from pre-injection arterial pressure to the maximum pressure achieved. The catecholamine injection is repeated every 10 minutes until three consecutive injections yield responses within 4 mmHg of each other. These three responses are then averaged and represent the control response to catecholamines. The test compound is suspended in 0.5% methylcellulose in water and is administered by gavage. The volume administered is 2 ml/kg body weight. Catecholamine bolus injections are given at 30, 45, 60, 75, 120, 150, and 180 minutes after gavage. The pressor response to the catecholamine is measured at each time point. The rats are then returned to their cage for future testing. A minimum of 3 days is allowed between tests. Percent inhibition is calculated for each time point following gavage by the following formula: ((Control Response—Response at time point)/Control Response)×100.

Assay “C”: Hypertensive Rat Model

Male rats are made hypertensive by placing a silver clip with an aperture of 240 microns on the left renal artery, leaving the contralateral kidney untouched. Sham controls undergo the same procedure but without attachment of the clip. One week prior to the surgery, animals to be made hypertensive are divided into separate groups and drug treatment is begun. Groups of animals are administered vehicle, endothelin receptor antagonists or ECE inhibitor alone, eplerenone alone, and combinations of endothelin receptor antagonists or ECE inhibitor and eplerenone at various doses:

Combination of

| Endothelin | | Endothelin | |
|---|---|---|---|
| receptor | | Receptor | |
| antagonist or | | Antagonist or | |
| ECE inhibitor | Eplerenone | ECE inhibitor | Eplerenone |
| (mg/kg/day) | (mg/kg/day) | (mg/kg/day) | (mg/kg/day) |
| 3 | 5 | 3 | 5 |
| | 20 | 3 | 20 |
| | 50 | 3 | 50 |
| | 100 | 3 | 100 |
| | 200 | 3 | 200 |
| 10 | 5 | 10 | 5 |
| | 20 | 10 | 20 |
| | 50 | 10 | 50 |
| | 100 | 10 | 100 |
| | 200 | 10 | 200 |
| 30 | 5 | 30 | 5 |
| | 20 | 30 | 20 |
| | 50 | 30 | 50 |
| | 100 | 30 | 100 |
| | 200 | 30 | 200 |

After 12 to 24 weeks, systolic and diastolic blood pressure, left ventricular end diastolic pressure, left ventricular dP/dt, and heart rate are evaluated. The hearts are removed, weighted, measured and fixed in formalin. Collagen content of heart sections are evaluated using computerized image analysis of picrosirius stained sections. It is expected that rats treated with a combination therapy of endothelin receptor antagonist or ECE inhibitor and eplerenone components, as compared to rats treated with either component alone, will show improvements in cardiac performance.

Assay “D”: Myocardial Infarction Rat Model:

Male rats are anesthetized and the heart is exteriorized following a left-sided thoracotomy. The left anterior descending coronary is ligated with a suture. The thorax is cloned and the animal recovers. Sham animals have the suture passed through without ligation. One-week prior to the surgery, animals to undergo infarction are divided into separate groups and drug treatment is begun. Groups of animals are administered vehicle, endothelin receptor antagonist or ECE inhibitor alone, eplerenone alone, and combinations of endothelin receptor antagonist or ECE inhibitor and eplerenone, at various doses, as follows:

Combination of

| Endothelin | | Endothelin | |
|---|---|---|---|
| receptor | | receptor | |
| antagonist or | | antagonist or | |
| ECE inhibitor | Eplerenone | ECE inhibitor | Eplerenone |
| (mg/kg/day) | (mg/kg/day) | (mg/kg/day) | (mg/kg/day) |
| 3 | 5 | 3 | 5 |
| | 20 | | 20 |
| | 50 | 3 | 50 |
| | 100 | 3 | 100 |
| | 200 | 3 | 200 |
| 10 | 5 | 10 | 5 |
| | 20 | 10 | 20 |
| | 50 | 10 | 50 |
| | 100 | 10 | 100 |
| | 200 | 10 | 200 |
| 30 | 5 | 30 | 5 |
| | 20 | 30 | 20 |
| | 50 | 30 | 50 |
| | 100 | 30 | 100 |
| | 200 | 30 | 200 |

After six weeks, systolic and diastolic blood pressure, left ventricular end diastolic pressure, left ventricular dP/dt, and heart rate are evaluated. The hearts are removed, weighed, measured and fixed in formalin. Collagen content of heart sections are evaluated using computerized image analysis of picrosirius stained sections. It is expected that rats treated with a combination therapy of endothelin receptor antagonist or ECE inhibitor, and eplerenone components, as compared to rats treated with either component alone, will show improvements in cardiac performance.

Therapy Protocols

Numerous well known, in vitro and in vivo testing schemes and protocols are useful to demonstrate the efficacy of aldosterone receptor antagonists and endothelin receptor antagonists and/or ECE inhibitors, both separately and in combination, for treating or preventing the pathological conditions. Non-limiting examples of testing schemes and protocols are described in references listed below, which are incorporated herein by reference:

Pitt, et al. NEJM 341, 709-717 (1999)

Pitt, et al. Cardiovasc Drug Ther. 15:79-87 (2001)

De Gasparo, et al. J Pharm Exp Ther 240, 650-656 (1986)

Blazer-Yost, et al. Am. J. Physiol 272, C1928-C1935 (1997)

Vijan, et al. J Gen Intern Med 12, 567-580 (1997)

Gentile, et al. Diabetes, Obesity and Metabolism 2, 355-362 (2000)

Sheng-Fang, et al. Am J Cardiol 86, 514-518 (2000)

Jick, et al. Lancet 356, 1627-1631 (2000)

Albert, et al. JAMA 286, 64-70 (2001)

Ridker, et al. NEJM 344, 1959-1965 (2001)

Wang, et al. JAMA 283, 3211-3216 (2000)

Meier, et al. JAMA 283, 3205-3210 (2000)

Biochem Biophys Res Commun 271, 688-692 (2000)

Mundy, et al. Science 286, 1946-1949 (1999)

Xiao, et al. J. Endocrinol 165, 533-536 (2000)

U.S. Pat. No. 5,730,992; U.S. Pat. No. 5,932,587; U.S. Pat. No. 6,180,597; Huval et al., WO00/69446; Huval et al., WO 00/69445; Cameron, WO 00/45818; Raza et al., WO 00/45817; Reszka, WO 99/66930; Scott, WO 99/11260; Delyani et al., WO 01/34132; and Alexander et al WO00/51642.

The following nonlimiting examples serve to illustrate the various aspects of the present invention.

### EXAMPLE 1:

Table 6 illustrates specific examples of the combinations of the present invention wherein the combination comprises an aldosterone receptor antagonist and an endothelin receptor antagonist, and wherein the aldosterone receptor antagonist and endothelin receptor antagonist together comprise a pharmacologically effective composition.

TABLE 6

| | Aldosterone Receptor | Endothelin |
|---|---|---|
| Combination | Antagonist Compound | Receptor |
| Number | Number of Table 1 | Antagonist |
| 1 | A-1 | bosentan |
| 2 | A-1 | sitaxsentan |
| 3 | A-1 | darusentan |
| 4 | A-1 | tezosentan |
| 5 | A-2 | bosentan |
| 6 | A-2 | sitaxsentan |
| 7 | A-2 | darusentan |
| 8 | A-2 | tezosentan |
| 9 | A-3 | bosentan |
| 10 | A-3 | sitaxsentan |
| 11 | A-3 | darusentan |
| 12 | A-3 | tezosentan |
| 13 | A-4 | bosentan |
| 14 | A-4 | sitaxsentan |
| 15 | A-4 | darusentan |
| 16 | A-4 | tezosentan |
| 17 | A-5 | bosentan |
| 18 | A-5 | sitaxsentan |
| 19 | A-5 | darusentan |
| 20 | A-5 | tezosentan |
| 21 | A-6 | bosentan |
| 22 | A-6 | sitaxsentan |
| 23 | A-6 | darusentan |
| 24 | A-6 | tezosentan |
| 25 | A-7 | bosentan |
| 26 | A-7 | sitaxsentan |
| 27 | A-7 | darusentan |
| 28 | A-7 | tezosentan |
| 29 | A-8 | bosentan |
| 30 | A-8 | sitaxsentan |
| 31 | A-8 | darusentan |
| 32 | A-8 | tezosentan |
| 33 | A-9 | bosentan |
| 34 | A-9 | sitaxsentan |
| 35 | A-9 | darusentan |
| 36 | A-9 | tezosentan |
| 37 | A-10 | bosentan |
| 38 | A-10 | sitaxsentan |
| 39 | A-10 | darusentan |
| 40 | A-10 | tezosentan |
| 41 | A-11 | bosentan |
| 42 | A-11 | sitaxsentan |
| 43 | A-11 | darusentan |
| 44 | A-11 | tezosentan |

Table 7 illustrates specific examples of the combinations of the present invention wherein the combination comprises an spironolactone and an endothelin receptor antagonist, and 5 wherein the spironolactone and endothelin receptor antagonist together comprise a pharmacologically effective composition.

TABLE 7

| | Aldosterone | Endothelin |
|---|---|---|
| Combination | Receptor | Receptor |
| Number | Antagonist | Antagonist |
| 1 | spironolactone | bosentan |
| 2 | spironolactone | sitaxsentan |
| 3 | spironolactone | darusentan |
| 4 | spironolactone | tezosentan |

### EXAMPLE 3:

Table 8 illustrates specific examples of the combinations of the present invention wherein the combination comprises eplerenone and bosentan, sitaxsentan, darusentan, or tezosentan, and wherein the combination of eplerenone and bosentan, sitaxsentan, darusentan, or tezosentan, together comprise a therapeutically effective composition for treating pathological conditions. The dosages of eplerenone and the identified endothelin receptor antagonist are provided in a dosage amount as herein described above.

TABLE 8

| | Aldosterone | Endothelin |
|---|---|---|
| Combination | Receptor | Receptor |
| Number | Antagonist | Antagonist |
| 1 | eplerenone | bosentan |
| 2 | eplerenone | sitaxsentan |
| 3 | eplerenone | darusentan |
| 4 | eplerenone | tezosentan |

The examples herein can be performed by substituting the generically or specifically described reactants and/or operating conditions of this invention for those used in the preceding examples.

In view of the above, it will be seen that the several aspects of the invention are achieved. As various changes could be made in the above methods, combinations and compositions of the present invention without departing from the scope of the invention, it is intended that all matter contained in the above description be interpreted as illustrative and not in a limiting sense. All documents mentioned in this application are expressly incorporated by reference as if fully set forth at length.

Definitions

To facilitate understanding of the invention, a number of terms as used herein are defined below:

“Combination therapy” means the administration of two or more therapeutic agents to treat a pathological condition in a subject, for example, the treatment of a pathological condition such as hypertension, cardiovascular disease, renal dysfunction, edema, cerebrovascular disease, or insulinopathy. Such administration encompasses co-administration of these therapeutic agents in a substantially simultaneous manner, such as in a single capsule having a fixed ratio of active ingredients or in multiple, separate capsules for each inhibitor agent. In addition, such administration encompasses use of each type of therapeutic agent in a sequential manner. In either case, the treatment regimen will provide beneficial effects of the drug combination in treating the pathological condition.

“ECE inhibitor” refers to any compound or mixture of compounds which inhibits the action of an endothelin converting enzyme from cleaving the Trp-Val bond in the precursor peptide big endothelin (Big ET).

“Endothelin receptor antagonist” refers to any compound or mixture of compounds which bind selectively or non-selectively to ET_{A} and/or ET_{B} receptors, the selective or non-selective binding thereby preventing endothelin isoforms ET-1, ET-2, and/or ET-3 from binding to ET_{A} and/or ET_{B} receptors.

“Epoxy-steroidal” is intended to embrace a steroidal nucleus having one or a plurality of epoxy-type moieties attached thereto.

“Pharmaceutically acceptable” is used adjectivally herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiologically acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

“Prophylaxis” and “prevention” include either preventing the onset of a clinically evident pathological condition altogether or preventing the onset of a preclinically evident stage of a pathological condition in a subject. These terms encompass, but are not limited to, the prophylactic treatment of a subject at risk of developing a pathological condition such as, but not limited to, hypertension, cardiovascular disease, renal dysfunction, edema, cerebrovascular disease, and insulinopathy.

“Steroidal”, as used in the phrase “epoxy-steroidal”, denotes a nucleus provided by a cyclopenteno-phenanthrene moiety, having the conventional “A”, “B”, “C” and “D” rings. The epoxy-type moiety may be attached to the cyclopentenophenanthrene nucleus at any attachable or substitutable positions, that is, fused to one of the rings of the steroidal nucleus or the moiety may be substituted on a ring member of the ring system.

“Subject” as used herein refers to an animal, preferably a mammal, and particularly a human, who has been the object of treatment, observation or experiment.

“Therapeutically-effective” qualifies the amount of each agent that will achieve the goal of improvement in pathological condition severity and the frequency of incidence over treatment of each agent by itself, while avoiding adverse side effects typically associated with alternative therapies.

“Treatment” refers to any process, action, application, therapy, or the like, wherein a subject, including a human being, is provided medical aid with the object of improving the subject's condition, directly or indirectly, or slowing the progression of a pathological condition in the subject.

When introducing elements of the present invention or the preferred embodiment(s) thereof, the articles “a”, “an”, and “the” are intended to mean that there are one or more of the elements. The terms “comprising”, “including” and “having” are intended to be inclusive and mean that there may be additional elements other than the listed elements.

What is claimed is:

1. A method for the prophylaxis or treatment of a pathological condition, the method comprising administering to a subject susceptible to or afflicted with such condition an aldosterone receptor antagonist and an endothelin receptor antagonist for the prophylaxis or treatment of a pathological condition, wherein the endothelin receptor antagonist is selected from compounds other than biphenyl sulfonamide compounds.

2. The method of claim 1 wherein the aldosterone receptor antagonist and endothelin receptor antagonist are simultaneously provided to the subject as part of a single composition.

3. The method of claim 1 wherein a first amount of the aldosterone receptor antagonist and a second amount of the endothelin receptor antagonist are provided to the subject in sequence as part of a timed relationship.

4. The method of claim 1 wherein the pathological condition is selected from the group consisting of hypertension, cardiovascular disease, renal dysfunction, liver disease, cerebrovascular disease, vascular disease, retinopathy, neuropathy, insulinopathy, edema, endothelial dysfunction, and baroreceptor dysfunction.

5. The method of claim 4 wherein the cardiovascular disease is selected from the group consisting of heart failure, arrhythmia, diastolic dysfunction, systolic dysfunction, ischemia, hypertrophic cardiomyopathy, sudden cardiac death, myocardial fibrosis, vascular fibrosis, impaired arterial compliance, myocardial necrotic lesions, vascular damage, myocardial infarction, left ventricular hypertrophy, decreased ejection fraction, cardiac lesions, vascular wall hypertrophy, endothelial thickening, and fibrinoid necrosis of coronary arteries.

6. The method of claim 4 wherein the renal dysfunction is selected from the group consisting of glomerulosclerosis, end-stage renal disease, diabetic nephropathy, reduced renal blood flow, increased glomerular filtration fraction, proteinuria, decreased glomerular filtration rate, decreased creatinine clearance, microalbuminuria, renal arteriopathy, ischemic lesions, thrombotic lesions, global fibrinoid necrosis, focal thrombosis of glomerular capillaries, swelling and proliferation of intracapillary cells, swelling and proliferation of extracapillary cells, expansion of reticulated mesangial matrix with or without significant hypercellularity, and malignant nephrosclerosis.

7. The method of claim 4 wherein the liver disease is selected from the group consisting of liver cirrhosis, liver ascites, and hepatic congestion.

8. The method of claim 4 wherein the cerebrovascular disease is stroke.

9. The method of claim 4 wherein the vascular disease is selected from the group consisting of thrombotic vascular disease, proliferative arteriopathy, atherosclerosis, decreased vascular compliance, and endothelial dysfunction.

10. The method of claim 4 wherein the insulinopathy is selected from the group consisting of insulin resistance, Type I diabetes mellitus, Type II diabetes mellitus, glucose resistance, pre-diabetic state, and syndrome X.

11. The method of claim 4 wherein the edema is selected from the group consisting of peripheral tissue edema, hepatic congestion, splenic congestion, liver ascites, respiratory congestion, and lung congestion.

12. The method of claim 1 wherein the aldosterone receptor antagonist is an epoxy-steroidal-type compound characterized in having a 9α,11α-substituted epoxy moiety.

13. The method of claim 1 wherein the aldosterone receptor antagonist is is selected from the group consisting of eplerenone and spironolactone.

14. The method of claim 1 wherein the aldosterone receptor antagonist is selected from the group consisting of:
pregn-4-ene-7,21-dicarboxylic acid, 9,11-epoxy-17-hydroxy-3-oxo,γ-lactone, methyl ester, (7α,11α,17α)-;
pregn-4-ene-7,21-dicarboxylic acid, 9,11-epoxy-17-hydroxy-3-oxo-dimethyl ester,(7α,11α,17α)-;
3′H-cyclopropa(6,7) pregna-4,6-diene-21-carboxylic acid, 9,11-epoxy-6,7-dihydro-17-hydroxy-3-oxo-,γ-lactone, (6β,7β,11α,17α)-;
pregn-4-ene-7,21-dicarboxylic acid, 9,11-epoxy-17-hydroxy-3-oxo-,7-(1-methylethyl) ester, monopotassium salt,(7α,11α,17α)-;
pregn-4-ene-7,21-dicarboxylic acid, 9,11,-epoxy-17-hydroxy-3-oxo-,7-methyl ester, monopotassium salt, (7α, 11α,17α) -;
3′H-cyclopropa(6,7)pregna-1,4,6-triene-21-carboxylic acid, 9,11-epoxy-6,7-dihydro-17-hydroxy-3-oxo-,g-actone(6α,7α,11α)-;
3′H-cyclopropa(6,7)pregna-4,6-diene-21-carboxylic acid, 9,11-epoxy-6,7-dihydro-17-hydroxy-3-oxo-,methyl ester, (6α,7α, 11α, 17α) -;
3′H-cyclopropa(6,7)pregna-4,6-diene-21-carboxylic acid, 9,11-epoxy-6,7-dihydro-17-hydroxy-3-oxo-,monopotassium salt, (6α,7α,11α,17α)-;
3′H-cyclopropa(6,7)pregna-4,6-diene-21-carboxylic acid, 9,11-epoxy-6,7-dihydro-17-hydroxy-3-oxo-,γ-lactone, (6α,7α,11α,17α)-;
pregn-4-ene-7,21-dicarboxylic acid, 9,11-epoxy-17-hydroxy-3-oxo-,γ-lactone, ethyl ester, (7α,11α,17α)-; and
pregn-4-ene-7,21-dicarboxylic acid, 9,11-epoxy-17-hydroxy-3-oxo-,γ-lactone, 1-methylethyl ester, (7α,11α,17α) -.

15. The method of claim 1 wherein the endothelin receptor antagonist is selected from the group consisting of bosentan, sitaxsentan, darusentan, tezosentan, enrasentan, tarasentan, atrasentan, ambrisentan, and the pharmaceutically acceptable salts, esters, conjugate acids, and prodrugs thereof.

16. The method of claim 15 wherein the aldosterone receptor antagonist is eplerenone.

17. The method of claim 1 wherein the aldosterone receptor antagonist is administered in a daily dose ranging from about 0.1 to 2000 mg, and the endothelin receptor antagonist is administered in a daily dose ranging from about 0.1 to 1000 mg.

18. The method of claim 1 further comprising administering a third amount of a compound selected from the group consisting of renin inhibitors, angiotensin I antagonists, angiotensin II antagonists, angiotensin converting enzyme inhibitors, alpha-adrenergic receptor blockers, beta-adrenergic receptor blockers, calcium channel blockers, endothelin receptor antagonists, endothelin converting enzyme inhibitors, vasodilators, diuretics, cyclooxygenase-2 inhibitors, apical sodium bile acid transport 10 inhibitors, cholesterol absorption inhibitors, fibrates, niacin, statins, cholesteryl ester transfer protein inhibitors, bile acid sequestrants, anti-oxidants, vitamin E, probucol, and IIb/IIIa antagonists.

19. The method of claim 1 further comprising administering a third amount of an ECE inhibitor.

20. The method of claim 19 wherein the aldosterone receptor antagonist is selected from the group consisting of eplerenone and spironolactone.

21. The method of claim 19 wherein the endothelin receptor antagonist is selected from the group consisting of bosentan, sitaxsentan, darusentan, tezosentan, enrasentan, tarasentan, atrasentan, ambrisentan, and the pharmaceutically acceptable salts, esters, conjugate acids, and prodrugs thereof.

22. The method of claim 19 wherein the ECE inhibitor is selected from the group consisting of CGS 26303, phosphoramidon, FR901533, TMC-66, SM-19712, SLV-306, KC-12615, KC-90095-1-AC, CGS-26303, CGS-30440, CGS-31447, CGS-26670, Sch-54470, and the pharmaceutically acceptable salts, esters, conjugate acids, and prodrugs thereof.

23. The method of claim 19 wherein the aldosterone receptor antagonist is is selected from the group consisting of eplerenone and spironolactone.

24. The method of claim 19 wherein the aldosterone receptor antagonist, endothelin receptor antagonist, and ECE enzyme inhibitor are simultaneously provided to the subject as part of a single composition.

25. The method of claim 19 wherein a first amount of the aldosterone receptor antagonist, a second amount of the endothelin receptor antagonist, and a third amount of an ECE enzyme inhibitor are provided to the subject in sequence as part of a timed relationship.

26. The method of claim 19 wherein the aldosterone receptor antagonist is administered in a daily dose ranging from about 0.1 to 2000 mg, the endothelin receptor antagonist is administered in a daily dose ranging from about 0.1 to 1000 mg, and the ECE inhibitor is administered in a daily dose ranging from about 0.1 to 1000 mg.

27. A combination comprising an aldosterone receptor antagonist and an endothelin receptor antagonist in a pharmaceutically acceptable carrier, wherein the endothelin receptor antagonist is selected from compounds other than biphenyl sulfonamide compounds.

28. The combination of claim 27 wherein the aldosterone receptor antagonist is selected from the group consisting of eplerenone and spironolactone.

29. A method for the prophylaxis or treatment of a pathological condition, the method comprising administering to a subject susceptible to or afflicted with such condition an aldosterone receptor antagonist and an ECE inhibitor for the prophylaxis or treatment of a pathological condition.

30. The method of claim 29 wherein the aldosterone receptor antagonist and ECE enzyme inhibitor are simultaneously provided to the subject as part of a single composition.

31. The method of claim 29 wherein a first amount of the aldosterone receptor antagonist and a second amount of the ECE enzyme inhibitor are provided to the subject in sequence as part of a timed relationship.

32. The method of claim 29 wherein the pathological condition is selected from the group consisting of hypertension, cardiovascular disease, renal dysfunction, liver disease, cerebrovascular disease, vascular disease, retinopathy, neuropathy, insulinopathy, edema, endothelial dysfunction, and baroreceptor dysfunction.

33. The method of claim 29 wherein the cardiovascular disease is selected from the group consisting of heart failure, arrhythmia, diastolic dysfunction, systolic dysfunction, ischemia, hypertrophic cardiomyopathy, sudden cardiac death, myocardial fibrosis, vascular fibrosis, impaired arterial compliance, myocardial necrotic lesions, vascular damage, myocardial infarction, left ventricular hypertrophy, decreased ejection fraction, cardiac lesions, vascular wall hypertrophy, endothelial thickening, and fibrinoid necrosis of coronary arteries.

34. The method of claim 29 wherein the renal dysfunction is selected from the group consisting of glomerulosclerosis, end-stage renal disease, diabetic nephropathy, reduced renal blood flow, increased glomerular filtration fraction, proteinuria, decreased glomerular filtration rate, decreased creatinine clearance, microalbuminuria, renal arteriopathy, ischemic lesions, thrombotic lesions, global fibrinoid necrosis, focal thrombosis of glomerular capillaries, swelling and proliferation of intracapillary cells, swelling and proliferation of extracapillary cells, expansion of reticulated mesangial matrix with or without significant hypercellularity, and malignant nephrosclerosis.

35. The method of claim 29 wherein the liver disease is selected from the group consisting of liver cirrhosis, liver ascites, and hepatic congestion.

36. The method of claim 29 wherein the cerebrovascular disease is stroke.

37. The method of claim 29 wherein the vascular disease is selected from the group consisting of thrombotic vascular disease, proliferative arteriopathy, atherosclerosis, decreased vascular compliance, and endothelial dysfunction.

38. The method of claim 29 wherein the insulinopathy is selected from the group consisting of insulin resistance, Type I diabetes mellitus, Type II diabetes mellitus, glucose resistance, pre-diabetic state, and syndrome X.

39. The method of claim 29 wherein the edema is selected from the group consisting of peripheral tissue edema, hepatic congestion, splenic congestion, liver ascites, respiratory congestion, and lung congestion.

40. The method of claim 29 wherein the aldosterone receptor antagonist is an epoxy-steroidal-type compound characterized in having a 9α,11α-substituted epoxy moiety.

41. The method of claim 29 wherein the aldosterone receptor antagonist is selected from the group consisting of eplerenone and spironolactone.

42. The method of claim 29 wherein the aldosterone receptor antagonist is selected from the group consisting of:
pregn-4-ene-7,21-dicarboxylic acid, 9,11-epoxy-17-hydroxy-3-oxo,γ-lactone, methyl ester, (7α,11α17α)-;
pregn-4-ene-7,21-dicarboxylic acid, 9,11-epoxy-17-hydroxy-3-oxo-dimethyl ester,(7α,11α,17α)-;
3′H-cyclopropa(6,7) pregna-4,6-diene-21-carboxylic acid, 9,11-epoxy-6,7-dihydro-17-hydroxy-3-oxo-,γ-lactone, (6β,7β,11β,17β)-;
pregn-4-ene-7,21-dicarboxylic acid, 9,11-epoxy-17-hydroxy-3-oxo-,7-(1-methylethyl)ester, monopotassium salt, (7α,11α,17α) -;
pregn-4-ene-7,21-dicarboxylic acid, 9,11,-epoxy-17-hydroxy-3-oxo-,7-methyl ester, monopotassium salt, (7α,11α,17α)-;
3′H-cyclopropa(6,7)pregna-1,4,6-triene-21-carboxylic acid, 9,11-epoxy-6,7-dihydro-17-hydroxy-3-oxo-,g-actone (6α,7α,11α) -;
3′H-cyclopropa(6,7)pregna-4,6-diene-21-carboxylic acid, 9,11-epoxy-6,7-dihydro-17-hydroxy-3-oxo-,methyl ester, (6α,7α,11α,17α)-;
3′H-cyclopropa(6,7)pregna-4,6-diene-21-carboxylic acid, 9,11-epoxy-6,7-dihydro-17-hydroxy-3-oxo-,monopotassium salt, (6α,7α,11α,17α)-;
3′H-cyclopropa(6,7)pregna-4,6-diene-21-carboxylic acid, 9,11-epoxy-6,7-dihydro-17-hydroxy-3-oxo-,γ-lactone, (6α,7α,11α,17α) -;
pregn-4-ene-7,21-dicarboxylic acid, 9,11-epoxy-17-hydroxy-3-oxo-,γ-lactone, ethyl ester, (7α,11α,17α)-; and
pregn-4-ene-7,21-dicarboxylic acid, 9,11-epoxy-17-hydroxy-3-oxo-,γ-lactone, 1-methylethyl ester, (7α,11α,17α) -.

43. The method of claim 29 wherein the ECE inhibitor is selected from the group consisting of CGS 26303, phosphoramidon, FR901533, TMC-66, SM-19712, SLV-306, KC-12615, KC-90095-1-AC, CGS-26303, CGS-30440, CGS-31447, CGS-26670, Sch-54470, and the pharmaceutically acceptable salts, esters, conjugate acids, and prodrugs thereof.

44. The method of claim 43 wherein the aldosterone receptor antagonist is is selected from the group consisting of eplerenone and spironolactone.

45. The method of claim 29 wherein the aldosterone receptor antagonist is administered in a daily dose ranging from about 0.1 to 2000 mg, and the ECE inhibitor is administered in a daily dose ranging from about 0.1 to 1000 mg.

46. The method of claim 29 further comprising administering a third amount of a compound selected from the group consisting of renin inhibitors, angiotensin I antagonists, angiotensin II antagonists, angiotensin converting enzyme inhibitors, alpha-adrenergic receptor blockers, beta-adrenergic receptor blockers, calcium channel blockers, ECE inhibitors, vasodilators, diuretics, cyclooxygenase-2 inhibitors, apical sodium bile acid transport inhibitors, cholesterol absorption inhibitors, fibrates, niacin, statins, cholesteryl ester transfer protein inhibitors, bile acid sequestrants, anti-oxidants, vitamin E, probucol, and IIb/IIIa antagonists.

47. A combination comprising an aldosterone receptor antagonist and an ECE inhibitor in a pharmaceutically acceptable carrier.

48. The combination of claim 47 wherein the aldosterone receptor antagonist is is selected from the group consisting of eplerenone and spironolactone.

49. A pharmaceutical composition comprising a first amount of an aldosterone receptor antagonist, a second amount of an endothelin receptor antagonist, and a pharmaceutically acceptable carrier, wherein the endothelin receptor antagonist is selected from compounds other than biphenyl sulfonamide compounds.

50. The composition of claim 49 wherein the first amount of the aldosterone receptor antagonist and the second amount of the endothelin receptor antagonist together comprise a therapeutically-effective amount of the aldosterone receptor antagonist and endothelin receptor antagonist for the prophylaxis or treatment of a pathological condition.

51. The composition of claim 50 wherein the aldosterone receptor antagonist is is selected from the group consisting of eplerenone and spironolactone.

52. The composition of claim 50 wherein the endothelin receptor antagonist is selected from the group consisting of bosentan, sitaxsentan, darusentan, tezosentan, enrasentan, tarasentan, atrasentan, ambrisentan, and the pharmaceutically acceptable salts, esters, conjugate acids, and prodrugs thereof.

53. The composition of claim 50 further comprising a third amount of a compound selected from the group consisting of renin inhibitors, angiotensin I antagonists, angiotensin II antagonists, angiotensin converting enzyme inhibitors, alpha-adrenergic receptor blockers, beta-adrenergic receptor blockers, calcium channel blockers, endothelin receptor antagonists, endothelin converting enzyme inhibitors, vasodilators, diuretics, cyclooxygenase-2 inhibitors, apical sodium bile acid transport inhibitors, cholesterol absorption inhibitors, fibrates, niacin, statins, cholesteryl ester transfer protein inhibitors, bile acid sequestrants, anti-oxidants, vitamin E, probucol, and IIb/IIIa antagonists.

54. The composition of claim 50 further comprising administering a third amount of an ECE inhibitor.

55. The composition of claim 54 wherein the aldosterone receptor antagonist is selected from the group consisting of eplerenone and spironolactone.

56. The composition of claim 54 wherein the endothelin receptor antagonist is selected from the group consisting of bosentan, sitaxsentan, darusentan, tezosentan, enrasentan, tarasentan, atrasentan, ambrisentan, and the pharmaceutically acceptable salts, esters, conjugate acids, and prodrugs thereof.

57. The composition of claim 54 wherein the ECE inhibitor is selected from the group consisting of CGS 26303, phosphoramidon, FR901533, TMC-66, SM-19712, SLV-306, KC-12615, KC-90095-1-AC, CGS-26303, CGS-30440, CGS-31447, CGS-26670, Sch-54470, and the pharmaceutically acceptable salts, esters, conjugate acids, and prodrugs thereof.

58. A pharmaceutical composition comprising a first amount of an aldosterone receptor antagonist, a second amount of an ECE inhibitor, and a pharmaceutically acceptable carrier.

59. The composition of claim 58 wherein the first amount of the aldosterone receptor antagonist and the second amount of the ECE inhibitor together comprise a therapeutically-effective amount of the aldosterone receptor antagonist and ECE inhibitor for the prophylaxis or treatment of a pathological condition.

60. The composition of claim 58 wherein the aldosterone receptor antagonist is selected from the group consisting of eplerenone and spironolactone.

61. The composition of claim 58 wherein the ECE inhibitor is selected from the group consisting of CGS 26303, phosphoramidon, FR901533, TMC-66, SM-19712, SLV-306, KC-12615, KC-90095-1-AC, CGS-26303, CGS-30440, CGS-31447, CGS-26670, Sch-54470, and the pharmaceutically acceptable salts, esters, conjugate acids, and prodrugs thereof.

62. The composition of claim 58 further comprising a third amount of a compound selected from the group consisting of renin inhibitors, angiotensin I antagonists, angiotensin II antagonists, angiotensin converting enzyme inhibitors, alpha-adrenergic receptor blockers, beta-adrenergic receptor blockers, calcium channel blockers, endothelin receptor antagonists, endothelin converting enzyme inhibitors, vasodilators, diuretics, cyclooxygenase-2 inhibitors, apical sodium bile acid transport inhibitors, cholesterol absorption inhibitors, fibrates, niacin, statins, cholesteryl ester transfer protein inhibitors, bile acid sequestrants, anti- oxidants, vitamin E, probucol, and IIb/IIIa antagonists.

63. A kit containing a first amount of an aldosterone receptor antagonist and a second amount of an endothelin receptor antagonist, wherein the endothelin receptor antagonist is selected from compounds other than biphenyl sulfonamide compounds.

64. The kit of claim 63 comprising the first amount of the aldosterone receptor antagonist in a unit dosage form, and the second amount of an endothelin receptor antagonist in a unit dosage form.

65. The kit of claim 63 wherein the aldosterone receptor antagonist is is selected from the group consisting of eplerenone and spironolactone.

66. The kit of claim 63 wherein the endothelin receptor antagonist is selected from the group consisting of bosentan, sitaxsentan, darusentan, tezosentan, enrasentan, tarasentan, atrasentan, ambrisentan, and the pharmaceutically acceptable salts, esters, conjugate acids, and prodrugs thereof.

67. The kit of claim 63 further comprising a third amount of an ECE inhibitor.

68. The kit of claim 67 wherein the endothelin receptor antagonist is selected from the group consisting of bosentan, sitaxsentan, darusentan, tezosentan, enrasentan, tarasentan, atrasentan, ambrisentan, and the pharmaceutically acceptable salts, esters, conjugate acids, and prodrugs thereof.

69. The kit of claim 67 wherein the angiotensin converting enzyme inhibitor is selected from the group consisting of CGS 26303, phosphoramidon, FR901533, TMC-66, SM-19712, SLV-306, KC-12615, KC-90095-1-AC, CGS-26303, CGS-30440, CGS-31447, CGS-26670, Sch-54470, and the pharmaceutically acceptable salts, esters, conjugate acids, and prodrugs thereof.

70. A kit containing a first amount of an aldosterone receptor antagonist and a second amount of an ECE inhibitor.

71. The kit of claim 70 comprising the first amount of the aldosterone receptor antagonist in a unit dosage form, and the second amount of an ECE inhibitor in a unit dosage form.

72. The kit of claim 70 wherein the aldosterone receptor antagonist is is selected from the group consisting of eplerenone and spironolactone.

73. The kit of claim 70 wherein the ECE inhibitor is selected from the group consisting of CGS 26303, phosphoramidon, FR901533, TMC-66, SM-19712, SLV-306, KC-12615, KC-90095-1-AC, CGS-26303, CGS-30440, CGS-31447, CGS-26670, Sch-54470, and the pharmaceutically acceptable salts, esters, conjugate acids, and prodrugs thereof.
